(19) United States
(12) Patent Application Publication
(10) Pub. No.: US 2005/0260208 A1
(43) Pub. Date: Nov. 24, 2005

(54) Binding agents and their use in targeting tumor cells

(75) Inventors: Hubert Eng, Edmonton (CA); Birgit C. Schultes, Arlington, MA (US)

Correspondence Address: FISH & NEAVE IP GROUP;ROPES & GRAY LLP, ONE INTERNATIONAL PLACE, BOSTON, MA, 02110-2624, US

(73) Assignee: AltaRex Medical Corp., Edmonton, AB (CA)

(21) Appl. No.: 10/510,361

(22) PCT Filed: Apr. 11, 2003
(86) PCT No.: PCT/US03/11457

§ 371 Date: Jun. 20, 2005

Related U.S. Application Data
(60) Provisional application No. 60/371,802, filed on Apr. 11, 2002.
(60) Provisional application No. 60/420,269, filed on Oct. 22, 2002.
(60) Provisional application No. 60/420,291, filed on Oct. 22, 2002.

(51) Int. Cl. A61K 39/395

(52) U.S. Cl. 424/155.1

(57) ABSTRACT

The present invention concerns methods and compositions for administering a binding agent to a patient wherein the patient generates a response to autologous tumor. The binding agents target apoptotic tumor cells and facilitates the uptake of these apoptotic tumor cell are taken up by dendritic cells or other antigen presenting cells for processing and presentation to the immune system without the expression of circulating tumor-associated antigen (or without the need of circulating tumor antigen).

### RELATED APPLICATIONS

This application claims the benefit of priority to U.S. provisional application 60/371,802, filed Apr. 11, 2002; to U.S. provisional application 60/420,269, filed Oct. 22, 2002; and to US provisional application 60/420,291, filed Oct. 22, 2002, all of which are hereby incorporated by reference in their entireties.

### BACKGROUND OF THE INVENTION

1. Technical Field

The present invention relates to the field of immunology and more particularly to the use of binding agents in combination with circulating tumor antigens or tumor cells and dendritic cells in promoting enhanced immunogenicity to autologous tumors.

2. Summary of the Related Art

T lymphocytes (i.e., T cells), unlike B lymphocytes (i.e., B cells), typically recognize their target antigen only when the antigen is presented in the context of the major histocompatibility complex (MHC). Thus, to present antigen to T lymphocytes, which include T helper cells and cytotoxic T cells, the antigen must be presented in context of an MHC molecule on the surface of an antigen presenting cell.

In particular, one type of antigen presenting cell, dendritic cells, has recently become of interest in the area of cancer immunotherapy. Dendritic cells are rare leukocytes that originate in the bone marrow and can be found distributed throughout the body (Steinman, Annu. Rev. Immunol. 9:271-296 (1991)), and are receiving increasing attention due to their potential inclusion as biological adjuvants in tumor vaccines (Bjork, Clinical Immunology 92: 119-127 (1999)). Dendritic cells express several receptors for the Fe portion of immunoglobulin IgG, which mediate the internalization of antigen-IgG complexes (ICs). In this capacity, dendritic cells are used to present tumor antigens to T cells. Several approaches have been adopted to directly load tumor antigens onto dendritic cells, including the pulsing of tumor peptides onto mature dendritic cells (Avigan, Blood Reviews 13: 51-64 (1999)). Isolated dendritic cells loaded with tumor antigen ex vivo and administered as a cellular vaccine have been found to induce protective and therapeutic anti-tumor immunity in experimental animals (Timmerman et al., Annu. Rev. Med. 50:507-529 (1999)).

European Patent No. EP0553244 describes an antigen/dual-specific binding agent complex for stimulating a response to the antigen, where the binding agent specifically binds both the antigen and a cell surface receptor on an antigen-presenting cell, but where binding of the binding agent to the cell surface receptor does not block the natural ligand for the receptor.

It has been found that antigen uptake by dendritic cells via Fcγ receptors results in functional augmentation of antigen presentation and T cell proliferation in an in vitro sheep system (Coughlan et al., Veterinary Immunology and Immunopathology 49: 321-330 (1996)). Further, Fcγ receptors induce dendritic cell maturation and promote efficient MHC class I-restricted presentation of peptides from exogenous, immunoglobulin (Ig) complexed antigens in the mouse system (Regnault et al., J. Exp. Med. 189: 371-380 (1999)).

Attempts have recently been made to utilized an ex vivo human model of myeloma to study the effects of ex vivo antibody/tumor cell complexes on dendritic cell uptake however the therapeutic benefit has not been established (Dhodpkar et al, J. Exp. Med. 195: 125-133 (2002)).

Thus, there remains a need to discover methods for utilizing dendritic cells to treat human diseases. The promise of dendritic cell-based approaches to treat disease such as cancer, underscores the need to actually develop such approaches as effective treatments.

### SUMMARY OF THE INVENTION

The present invention provides effective therapeutic methods, compositions, and pharmaceutical packages for treatment of diseases associated with tumor cells.

The compositions according to the invention comprise binding agents, dendritic cells, tumor cell antigens, tumor cells, apoptotic tumor cells, binding agent-tumor cell antigen complexes, and apoptosis-inducing agents. The compositions according to the invention can be generated ex vivo and administered to a patient or administered directly to a patient for an in vivo therapeutic effect. Administration of the compositions of the present invention can be done in the presence or absence of the following: adjuvants, immunogenic carriers, and apoptosis-inducing agents. The compositions according to the invention are effective when administered to a patient at a dose of less than about 2 mg per patient.

One aspect of the present invention provides for a method for treating a patient to reduce proliferation of and/or kill target cells that express a multiepitopic antigen, comprising administering one or more agents that cause apoptosis of the target cells; and administering an antibody immunoreactive with said multiepitopic antigen, which antibody can induce an anti-idiotypic response to said multiepitopic antigen, and said antibody is cytotoxic to said target cells which is accessible on target cells undergoing apoptosis and said antibody induces endocytosis of the apoptotic target cell by an antigen-presenting cell. The target cells are transformed cells (e.g., tumor cells). The method of the present invention reduces the number of target cells in the patient. The compositions of the present invention can be administered separately or conjointly. The one or more agents that cause apoptosis of the target cells of the present invention are chemotherapeutic agents. Antibodies of the present invention include, for example, xenotypic monoclonal antibodies, such as Alt-1, Alt-2, Alt-3, Alt-4, and Alt-5. When administered to a patient in need thereof, compositions of the present invention elicit an effective B cell and/or T cell response when administered to the patient, wherein the effective T cells response is a T helper response; a CTL response; or a T helper response and a CTL response. Preferably, the patient of the present invention is a human patient

One embodiment of the present invention is a packaged pharmaceutical for treating a patient to reduce proliferation of and/or kill target cells that express a multiepitopic antigen, comprising an antibody formulation immunoreactive with said multiepitopic antigen, which is accessible on target cells undergoing apoptosis and said antibody induces endocytosis of the apoptotic target cell by an antigen presenting cell can induce an anti-idiotypic response to said multiepitopic antigen, and said antibody is cytotoxic to said target cells; and instructions for using the antibody in conjunction with a treatment that causes apoptosis of the target cells. The packaged pharmaceutical can further comprise one or more agents that cause apoptosis of the target cells, such as a chemotherapeutic agent. The compositions of the packaged pharmaceutical can be formulated separately from, or with, the antibody. The antibody of the packaged pharmaceutical is preferably a xenotypic monoclonal antibody, such as Alt-1, Alt-2, Alt-3, Alt-4, and Alt-5. Target cells of the packaged pharmaceutical can be a transformed cell, such as a tumor cell. The one or more agents that cause apoptosis of target cells and the antibody of the packaged pharmaceutical induce an effective B cell and/or T cell response in the patient, wherein the effective T cell response is a T helper response; a CTL response; or a T helper response and a CTL response. The compositions of the pharmaceutical package can be formulated at a low dose wherein patients receive a 2 mg dose or less. Examples of lower formulations include, for example, a dosage of about 100 μg/patient to about 2 mg/patient; or a dosage of about 0.1 μg/patient to about 200 μg/patient.

One embodiment of the present invention provides for a kit for treating a patient to reduce proliferation of and/or kill target cells that express a multiepitopic antigen, comprising one or more agents that cause apoptosis of the target cells ex vivo; an antibody formulation immunoreactive with said multiepitopic antigen, which is accessible on target cells undergoing apoptosis and said antibody induces endocytosis of the apoptotic target cell by an antigen presenting cell can induce an Ab3′ response to said multiepitopic antigen, and said antibody and Ab3′ response are cytotoxic to said target cells; and instructions for treating target cells ex vivo with said apoptotic agent(s) and administering treated target cells conjointly with said antibody formulation. The kit of the present invention can further include a means for isolating target cells from a patient sample. Such means include an affinity purification means, such as an antibody; a lectin; a His-tag; and an enterokinase cleavage tag. The kit of the present invention can further include a means for isolating dendritic cells or other antigen-presenting cells from a patient sample. Such means include an affinity purification means, such as an antibody or a lectin; magnetic beads, adhesion surfaces or an elutriation machine The antibody of the kit is preferably a xenotypic monoclonal antibody, such as Alt-1; Alt-2; Alt-3; Alt-4; and Alt-5. The one or more agents that cause apoptosis of the target cells ex vivo as provided in the kit can be a chemotherapeutic agent.

### BRIEF DESCRIPTION OF THE DRAWINGS

FIG. 1: Time course of apoptosis.

FIG. 1A: Time course of cell death in NIH:OVCAR-3 cells treated with chemotherapeutics.

FIG. 1B: Time course of apoptosis-related Annexin V increase.

FIG. 2: Expression on tumor cells CA125 expression on tumor cells (NIH:OVCAR-3) either untreated or treated with Taxol.

FIG. 2A: Annexin V staining on CA125 positive cells which are untreated.

FIG. 2B: Annexin V staining on CA125 positive cells which are treated with Taxol.

FIG. 2C: A comparison of Annexin V staining on CA125 positive cells which are either untreated or treated with a variety of chemotherapeutic agents.

FIG. 3: Illustration of ex vivo approach and increased tumor lysis from dendritic cells loaded with tumor cells rendered apoptotic via gamma irradiation, MAb-B43.13 or apoptotic tumor cells plus B43.13 to stimulate T cells. Tumor cell lysis by activated T cells is measured by Chromium release assay.

FIG. 4: Illustration of ex vivo approach and increased tumor lysis from dendritic cells loaded with tumor cells rendered apoptotic. Tumor cell lysis by activated T cells is measured by Chromium release assay.

FIG. 4A: Illustration of ex vivo approach and increased tumor lysis from the administration of dendritic cells loaded with tumor cells rendered apoptotic via Taxol or controls, MAb-B43.13 or apoptotic tumor cells plus B43.13 to stimulate T cells.

FIG. 4B: Illustration of ex vivo approach and increased tumor lysis from the administration of dendritic cells loaded with tumor cells rendered apoptotic via doxorubicin or controls, MAb-B43.13 or apoptotic tumor cells plus B43.13 to stimulate T cells.

FIG. 5: Illustration of tumor cell lysis by T cells stimulated with dendritic cells (DC) loaded with apoptotic tumor cells (4 h after chemotherapy or irradiation) or necrotic tumor cells (repeated freeze-thaw) or negative control with and without addition of the binding agent B43.13.

FIG. 6: Illustration of interferon-gamma production by T cells stimulated with dendritic cells (DC) loaded with apoptotic tumor cells (4 h after Taxol or irradiation treatment) with and without addition of the binding agent B43.13.

FIG. 7: Illustration of in vivo approach and enhanced T cell activity against autologous tumor in patients administered MAb-B43.13 prior to with chemotherapy as measured by ELISPOT with a baseline measurement and at week 12.

FIG. 8: Illustration of in vivo approach and enhanced T cell activity against CA125 and autologous tumor in patients administered MAb-B43.13 prior to (Week 12) and after chemotherapy (Week 26) as measured by ELISPOT.

FIG. 8A illustrates the experiment wherein autologous dendritic cells were loaded with CA125 and incubated with patients' T cells in the last 24 hours of culture.

FIG. 8B illustrates the experiment wherein autologous dendritic cells were loaded with tumor cells and incubated with patients' T cells in the last 24 hours of culture.

FIG. 9: Illustration of in vivo approach using a Kaplan Meier representation of a correlation between the treatment effect as measured by survival and T cell activity.

FIG. 9A: Illustration of in vivo approach using a Kaplan Meier representation of a correlation between the treatment effect as measured by time to progression and T cell activity against autologous tumor and/or CA125.

FIG. 9B: Illustration of in vivo approach using a Kaplan Meier representation of a correlation between the treatment effect as measured by survival and T cell activity against autologous tumor and/or CA125.

### DISCLOSURE OF THE INVENTION

### I. Overview

Many chemotherapeutic agents are cytotoxic, and their effectiveness in treating cancer is based upon the fact that cancerous cells are generally more sensitive to such cytotoxic therapies than are normal cells either because of their rapid metabolism, or because they employ biochemical pathways not employed by normal cells. For many chemotherapeutics, cytotoxic effects are thought to be the consequence of inducing programmed cell death, also referred to as apoptosis. However, a major obstacle in chemotherapy can be the development of chemoresistance, which reduces or negates the effectiveness of many chemotherapeutic agents. Such resistance is often linked to the inability of the chemotherapeutic agents to induce apoptosis in particular cancer cells. Counteracting chemoresistance can restore efficacy of many chemotherapeutic agents, and can help lower the dosage of these agents, thereby alleviating or avoiding unwanted side effects of these agents.

Chemotherapy, however, is not specific to tumor cells, but also destroys other proliferating cells such as blood cells. These include cells of the immune system like activated B and T cells. Therefore, it is widely believed that chemotherapy would not be synergistic with vaccine approaches.

The invention relates to immunotherapy. More particularly, the invention relates to the use of binding agents and antigen presenting cells, in particular dendritic cells, in immunotherapy. The invention provides a therapeutically effective tumor cell-based approach to the treatment of cancer. The patents and publications cited herein and are hereby incorporated by reference in their entirety.

The invention provides methods and compositions for treating a patient suffering from cancer. The methods and compositions according to the invention comprise combining ex vivo or in vivo a binding agent specific for an antigen on an apoptotic tumor cell, the apoptotic tumor cell and a dendritic cell, wherein the patient receives a therapeutic benefit.

If a specific antibody from one animal species is injected as an immunogen into a suitable second species, the injected antibody will elicit an immune response (e.g., produced antibodies or T cells against the injected antibodies—“anti-antibodies”). A xenotypic antibody is therefore believed to be more immunogenic and more beneficial to induce an immune response to an otherwise not recognized antigen compared to an antibody from the same species. Some of these anti-antibodies will be specific for the unique epitopes (i.e., idiotopes) of the variable domain of the injected antibodies. These epitopes are the idiotype of the primary antibody; thus the secondary (anti-) antibodies which bind to these epitopes are anti-idiotypic antibodies. The sum of all idiotopes present on the variable portion of an antibody is its idiotype. The Ab2 have binding site that is the complement of the original antigen, and thus, will reproduce the “internal image” of the original antigen and acts as a surrogate antigen.

Antibodies produced initially during an immune response will carry unique epitopes to which the organism is not tolerant, and therefore, will elicit production of secondary antibodies (Ab2) directed against the idiotypes of the primary antibodies (Ab1). The Ab2, in turn, has an idiotype which induces induction of tertiary antibodies (Ab3).
Ab1→Ab2→Ab3

The present invention involves an antibody immunoreactive with a pre-determined epitope of a multiepitopic target cell-associated antigen, which is accessible on target cells undergoing apoptosis and said antibody induces endocytosis of the apoptotic target cell by an antigen-presenting cell. This that alters the recognition of the target cell antigen in a manner such that the host immune system can recognize and initiate an immune response to the previously unrecognized target cell. Such immune response can include antibodies, T helper cells and/or cytolytic T cells specific for the target,cell antigen. One salient feature of this invention is the production of Ab3′ antibodies that recognize a second epitope on the multiepitopic antigen such that the Ab3′ (anti-idiotypic) antibodies bind a second epitope on the antigen that is exposed once the antigen is altered.

### II. Exemplary Definitions

As used herein the term “species” or “animal” refers to mammals, preferably mammals such as humans. Likewise, a “patient” or “subject” to be treated by the method of the invention can mean either a human or non-human animal.

“immunogenic complex” as used herein means a binding agent/tumor target cell complex that was not recognized by the immune system prior to the in vivo or ex vivo binding linking of the binding agent to a tumor target cell antigen on a tumor target cell or a to circulating tumor cell antigen.

A “binding agent”, as used herein, refers to one member of a binding pair, including an immunologic pair, e.g., a binding moiety that is capable of binding to an antigen, preferably but not limited to a single epitope expressed on the antigen, such as a predetermined tumor antigen. In some embodiments of the invention, the binding agent, when bound to the antigen, forms an immunogenic complex. In one embodiment, the binding agents encompass antibodies.

The term “antibody” as used herein, unless indicated otherwise, is used broadly to refer to both antibody molecules and a variety of antibody-derived molecules. Such antibody derived molecules comprise at least one variable region (either a heavy chain of or a light chain variable region), as well as individual antibody light chains, individual antibody heavy chains, chimeric fusions between antibody chains and other molecules, and the like. Functional immunoglobulin fragments according to the present invention may be Fv, scFv, disulfide-linked Fv, Fab, and F(ab′)2. Antibodies, or fragments thereof, of the present invention, can be cytotoxic to target cells such that they induce antibody dependent cellular cytotoxicity (ADCC) or complement dependent cytotoxicity (CDC) but are not required to.

Also encompassed by the term “antibody” are polyclonal antibodies, monoclonal antibodies (“MAb”), preferably IgG1 antibodies; chimeric monoclonal antibodies (“C-MAb”); humanized antibodies; genetically engineered monoclonal antibodies (“G-MAb”).

The antibody may be a “bispecific antibody” which has two binding sites, one that is specific for the (apoptotic) tumor cell of the invention and the other that is specific for the receptor, e.g., at its ligand-binding site, on the surface of a dendritic cell. In certain preferred embodiments, the binding agent of the invention is an antibody where the binding site is specific for the target cell antigen and the constant region or carbohydrate portion are responsible for receptor engagement, e.g. the ligand site. Preferably the antibody is provided at a concentration of from about 1002 mg/patient or 1-100 μg/kg10 pg/ml.

“An active portion of an antibody” is a molecule that includes a tumor target cell binding site that is specific for a tumor target cell antigen. Alternatively, an “active portion of an antibody” is a molecule that includes a receptor binding site that binds a receptor on dendritic cells with its ligand-binding site (e.g., the Fc portion of the antibody including the heavy chain constant region or the carbohydrate chain at the hinge region). Accordingly, an antibody of the invention may be, e.g., chimeric, single chain, mutant, or antibody fragment so long as the antibody is able to specifically bind a tumor cell and so long as the antibody includes a portion that binds a receptor on the dendritic cell with its ligand-binding site while the target cell is bound.

Preferred binding agents of the invention are monoclonal antibodies, and even more preferably, xenotypic monoclonal antibodies. Where the patient is human, these xenotypic monoclonal antibodies include, without limitation, murine monoclonal antibodies. Particularly preferred murine monoclonal antibodies include Alt-1 (murine IgG1, specifically binds to MUC-1; ATCC No. PTA-975; American Type Culture Collection, Manassas, Va.), Alt-2 (OvaRex MAb B43.13, oregovomabmurine IgG1, specifically binds to CAI CA125; ATCC No. PTA-1883), Alt-3 (murine IgG3, specifically binds to CAI CA19.9; ATCC No. PTA-2691), Alt-4 (murine IgM, specifically binds to CA19.9; ATCC No. PTA-2692), and Alt-5 (murine IgG 1, specifically binds to CAI CA19.9; ATCC No. PTA-2690); and Alt-6 (murine IgG1, specifically binds to prostate specific antigen (PSA); ATCC No. HB-12526).

In one embodiment of the present invention, a binding agent encompasses antigen-binding peptides; tumor-binding peptides; a protein, including receptor-specific proteins; a peptide binding to a receptor, a carbohydrate binding to a receptor; a polypeptide; a glycoprotein; a lipoprotein (e.g., growth factors); lymphokines and cytokines; enzymes, immune modulators; hormones (e.g., somatostatin); any of the above joined to a molecule that mediates an effector function; and mimics or fragments of any of the above. The binding agents of the present invention may be labeled or unlabeled. Binding agents of the present invention can be further engineered to create a fusion protein wherein the first portion of the fusion protein contains a portion that binds to the tumor target cell antigen as described above, and the second portion of the fusion protein contains an Fc portion, complement-fixing components or carbohydrates that is are capable of binding to a receptor on a dendritic cell.

As used herein, “immunoreactive” refers to binding agents, antibodies or fragments thereof that are specific to a tumor target cell antigen, yet if are cross-reactive to other proteins, are not toxic at the levels at which they are formulated for administration to human use. “Specifically binds” means that the binding agent binds to the antigen on the target cell with greater affinity than it binds unrelated antigens. Preferably such affinity is at least 10-fold greater, more preferably at least 100-fold greater, and most preferably at least 1000-fold greater than the affinity of the binding agent for unrelated antigens. The terms “immunoreactive” and “specifically binds” are used interchangeably herein.

“Administering” is defmed herein as a means providing the composition to the patient in a manner that results in the composition being inside the patient's body. Such an administration can be by any route including, without limitation, subcutaneous, intradermal, intravenous, inta-arterial, intraperitoneal, and intramuscular. Compositions of the present invention can be administered conjointly (e.g., in the same formulation, or in different formulations administered at the same time) or administered separately.

A physician or veterinarian having ordinary skill in the art can readily determine and prescribe the “effective amount” (ED₅₀) of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The phrase “therapeutically effective amount” as used herein means that amount of a compound, material, or composition comprising a compound of the present invention which is effective for producing some desired therapeutic effect by inducing tumor-specific immune responses of tumor cells in a patient and thereby blocking the biological consequences of that pathway in the treated cells eliminating the tumor cell or preventing it from proliferating, at a reasonable benefit/risk ratio applicable to any medical treatment

An “effective immune response” is defined herein wherein the patient experiences partial or total alleviation or reduction of signs or symptoms of illness, and specifically includes, without limitation, prolongation of survival. The patient's symptoms remain static, and the tumor burden does not increase. Further, an effective immune response is an effective B and/or T cell response. The T cell response can be a T helper response, a CTL response, or both a T helper and a CTL response.

“Induction of a B cell response” is defined herein as causing production of tumor cell-specific antibodies.

“Induction of CTL” is defined herein as causing potentially cytotoxic T lymphocytes to exhibit tumor cell specific cytotoxicity.

“Tumor cell specific antibody” is defined herein as the ability of the antibody to specifically bind to the target cell. As used herein, the specificity of the antibody for a tumor cell can be measured wherein the affinity of the antibody to the tumor cell is greater then to other cells not associated with the tumor.

“Tumor cell specific cytotoxicity” is defined herein as the ability of the cytotoxic T lymphocyte to specifically kill the target cell. As used herein, the specificity of a CTL for a tumor cell can be measured wherein cytotoxicity against a tumor cell associated with the disease is greater than a cell that is not associated with the tumor.

“Induction of a T helper response” is defined herein as causing T helper cells to provide the support to B cells or CTL such that an effective antibody or cytolytic response is induced.

Each of the embodiments of the present invention can be used as a composition when combined with a pharmaceutically acceptable carrier or excipient. “Carrier” and “excipient” are used interchangeably herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier” is defined herein as a carrier that is physiologically acceptable to the administered patient and that retains the therapeutic properties of the dendritic cell binding agent and apoptotic tumor cell (and/or dendritic cell) with which it is administered. Pharmaceutically-acceptable carriers and their formulations are well-known and generally described in, for example, Remington's pharmaceutical Sciences (18^{th} Edition, ed. A. Gennaro, Mack Publishing Co., Easton, Pa., 1990). On exemplary pharmaceutically acceptable carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject binding agents or treated dendritic cells from the administration site of one organ, or portion of the body, to another organ, or portion of the body. Each carrier must be “acceptable” in the sense of being compatible with the other ingredients of the formulation and not injurious to the patient. Nor should a pharmaceutically acceptable carrier alter the specific activity of the binding agents of treated dendritic cells. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “tumor cell antigen” is defined herein as an antigen that is present in higher quantities on a tumor cell or in body fluids than unrelated tumor cells, normal cells, or in normal body fluid. The antigen presence may be tested by any number of assays known to those skilled in the art and include without limitation negative and/or positive selection with antibodies, such as an ELISA assay, a Radioinmmunoassay, or by Western Blot.

As used herein, the term “cancer” is used to mean a condition in which a cell in a patient's body undergoes abnormal, uncontrolled proliferation. Non-limiting examples of cancers include leukemias, multiple myelomas, prostate, ovarian, testicular, breast, or lung tumor, melanomas, lymphomas, etc. As used herein, the term “cancer” refers to any neoplastic disorder, including such cellular disorders as, for example, renal cell cancer, Kaposi's sarcoma, chronic leukemia, breast cancer, sarcoma, ovarian carcinoma, rectal cancer, throat cancer, melanoma, colon cancer, bladder cancer, mastocytoma, lung cancer, mammary adenocarcinoma, pharyngeal squamous cell carcinoma, gastrointestinal or stomach cancer, epithelial cancer, or pancreatic cancer.

As used herein, “transformed cells” refers to cells that have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. For purposes of this invention, the terms “transformed phenotype of malignant mammalian cells” and “transformed phenotype ” are intended to encompass, but not be limited to, any of the following phenotypic traits associated with cellular transformation of mammalian cells: immortalization, morphological or growth transformation, and tumorigenicity, as detected by prolonged growth in cell culture, growth in semi-solid media, or tumorigenic growth in immuno-incompetent or syngeneic animals.

By “treating” a patient suffering from cancer it is meant that the patient's symptoms are partially or totally alleviated, or remain static following treatment according to the invention. A patient that has been treated can exhibit a partial or total alleviation of symptoms and/or tumor load. The term “treatment” is intended to encompass prophylaxis, therapy and cure.

The term “sample” is defined herein as blood, blood product, biopsy tissue, serum, and any other type of fluid or tissue that can be extracted from a patient suffering from cancer that would contain tumor cells, or tumor cell antigens thereof, and dendritic cells.

By “combining” ex vivo means bringing into physical proximity outside of the body. “Combining” and “contacting” are used interchangeably herein and are meant to be defined in the same way.

“Allogeneic” is defined herein as cells originating from a source other than the patient, such as from an existing cell bank (e.g., NIH: OVCAR-3 cell line) or a donor or other source not originating from the patient.

“Autologous” is defined herein as cells originating from a patient wherein the cells have identically matched MHC loci (both class I and class II). Thus, an identical sibling can provide autologous dendritic cells for a patient. Similarly, a close relative can provide autologous dendritic cells for a patient, so long as the patient and the close relative have identically matched MHC loci. Of course, two individuals of an inbred strain of laboratory animal (e.g., inbred BALB/c mice) are autologous to one another.

The terms “apoptosis” or “programmed cell death,” refers to the physiological process by which unwanted or useless cells are eliminated during development and other normal biological processes. Apoptosis, is a mode of cell death that occurs under normal physiological conditions and the cell is an active participant in its own demise (“cellular suicide”). It is most often found during normal cell turnover and tissue homeostasis, embryogenesis, induction and maintenance of immune tolerance, development of the nervous system and endocrine-dependent tissue atrophy. Cells undergoing apoptosis show characteristic morphological and biochemical features. These features include chromatin aggregation, nuclear and cytoplasmic condensation, partition of cytoplasm and nucleus into membrane bound vesicles (apoptotic bodies), which contain ribosomes, morphologically intact mitochondria and nuclear material. In vivo, these apoptotic bodies are rapidly recognized and phagocytized by either macrophages, dendritic cells or adjacent epithelial cells. Due to this efficient mechanism for the removal of apoptotic cells in vivo no inflammatory response is elicited. In vitro, the apoptotic bodies as well as the remaining cell fragments ultimately swell and finally lyse. This terminal phase of in vitro cell death has been termed “secondary necrosis.” Apoptosis can be measured by methods known to those skilled in the art like DNA fragmentation, exposure of Annexin V, activation of caspases, release of cytochrome c, etc. A tumor cell that has been induced to die is termed herein as an “apoptotic tumor cell”.

“Recognized” as used herein means that the immune system was not responsive inactivated (e.g., absence of a B or T cell response to the tumor cell) and after administration, a B and/or T cell immune response is elicited that targets the induces apoptosis of a tumor cell).

“Apoptosis inducing agent” is defined herein to induce apoptosis/programmed cell death, and include, for example, irradiation, chemotherapeutic agents or receptor ligation agents, wherein the tumor cells are induced to undergo programmed cell death. Some non-limiting examples of “chemotherapeutic agents” include (liposomal) rubicin, doxobucin, taxans, topoisomerase inhibitors, carboplatin, and cisplatin. “Irradiation” as used herein means to treat the tumor cells by using standard radiation treatment and including but not limited to γ irradiation. “Receptor ligation” as used herein means to treat the tumor cells by using antibodies or ligands to receptors that trigger induction of apoptosis such as the receptors of the EGF receptor family or CD20.

A “dendritic cell” is defined herein as a bone marrow-derived cell that can internalize antigen and process the antigen such that it (or a peptide derived from an antigen of the tumor cell) is presented in the context of both the MHC class I complex and the MHC class II complex. Accordingly, a dendritic cell of the invention is able to activate both CD8+ T cells (which are primarily cytotoxic T lymphocytes) and CD4+ T cells (which are primarily helper T cells). It should be understood that any cell capable of presenting a peptide derived from an internalized antigen on both class I and class II MHC is a dendritic cell of the invention. Preferably, a dendritic ell of the invention has the phenotype and characteristics of the dendritic cells described in Steinman, Annu. Rev. Immunol. 9: 271-296 (1991).

“Immature dendritic cells” are defined herein as a population of dendritic cells having preferably one or more of the cell surface antigens at the indicated level of expression as described in PCT application WO 01/85204 by Schultes et al.

“Precursor dendritic cells” are defined herein as a population of cells, each of which is capable of becoming a dendritic cell, e.g. monocytes, where greater than 80% of the population have CD64 and CD32 antigen present and about 70% of the population is positive for CD14.

Human dendritic cells preferably express the cell surface molecules described below in Table I at its different maturation stages. Note that expression of the Fc receptors, particularly the CD64 (FCγRI) typically decreases as the dendritic cell matures.

TABLE I
Human Dendritic Cell Surface Markers

| | Day 0 | Day 4 | Day 7 |
|---|---|---|---|
| Marker (all cells) | Monocytes | Immature | Matur Dendritic Cell |
| | | Dendritic Cell | |
| HLA-DR | 70-85% | 80-85% | 95-99% |
| HLA-ABC | 70-85% | 85-90% | 95-99% |
| CD3 | 1-5% | ND | ND |
| CD4 | 2-3% | ND | ND |
| CD8 | 2-3% | ND | ND |
| CD16 | 3-15% | 15-40% | 0.5-5% |
| CD19 | 5-10% | ND | ND |
| CD14 | 75-80% | 0.4-0.5% | 0.1-0.2% |
| CD1 is | 75-80% | 95-99% | 99-100% |
| Marker (gated on | | | |
| dendritic cells) | | | |
| Cells | | | |
| CD86 | 85-90% | 40-70% | 95-99% |
| CD80 | 30-50′/0 | 55-80% | 85-90% |
| CD40 | 40-50% | 55-60% | 55-60% |
| CD83 | 10-15% | 10-15% | 55-60% |
| CD32 | 89-98% | 70-95% | 40-45% |
| CD64 | 92-99% | 28-60% | 4-10% |

### III. Exemplary Embodiments

A. Compounds and Compositions

In one aspect, a composition comprises a binding agent. In a further embodiment, the binding agent is an antibody, and additionally, can be a xenotypic monoclonal antibody. Specific examples of xenotypic monoclonal antibodies include, for example, Alt-1 (murine IgG1, specifically binds to MUC-1; ATCC No. PTA-975; American Type Culture Collection, Manassas, Va.), Alt-2 (OvaRex® MAb B43.13, oregovomab, murine IgG1, specifically binds to CAI CA125; ATCC No. PTA-1883), Alt-3 (murine IgG3, specifically binds to CAI CA19.9; ATCC No. PTA2691), Alt-4 (murine IgM, specifically binds to CA19.9; ATCC No. PTA-2692), and Alt-5 (murine IgG 1, specifically binds to CAI CA19.9; ATCC No. PTA-2690).

In a further embodiment, the composition further comprises a tumor cell, or tumor cell antigen thereof, obtained from a sample from a patient, whereby a binding agent is immunogenic with the tumor cell antigen. The tumor cell can be alive (i.e., non-apoptotic), wherein the tumor cell can be treated ex vivo with an apoptotic-inducing agent. Alternatively, the tumor cell can be apoptotic, where apoptosis has been induced in vivo by irradiation, chemotherapy or receptor ligation. In a further embodiment, the binding agent and tumor cell, or tumor cell antigen thereof are contacted ex vivo and administered to a patient as a complex

In a further invention, the antibody-apoptotic tumor cell complex can be affinity purified prior to administration to the patient. Affinity purification can be accomplished by use of a His-tag sequence, an enterokinase cleavage tag, or a magnetic bead system. Thus, enriched complexes can be administered to the patient.

The compositions according to the invention are useful for providing a therapeutic benefit to patients suffering from cancer. A transformed cell may proliferate to form a solid tumor, or may proliferate to form a multitude of cells (e.g., leukemia). Preferably, the cancer of the invention is metastatic. Note that because cancer is the abnormal, uncontrolled proliferation of a patient's cell, the term does not encompass the normal proliferation of a cell, such as a stem cell or a spermatocyte.

In certain embodiments the composition may be obtained by combining ex vivo the binding agent, the apoptotic tumor cell, and an autologous dendritic cell. The apoptotic tumor cells may be allogenic or autologous and inactivated by treatment with a chemotherapeutic agent, irradiation, or receptor ligation.

In further embodiments, the composition further comprises a dendritic cell. Preferably, the dendritic cell is autologous to the patient. In preferred embodiments the composition contains at least one dendritic cell, more preferably the composition contains a concentration of 10⁵ to 10⁸ dendritic cells per patient per treatment. Isolation of dendritic cells or other antigen-presenting cells from a patient sample can be accomplished by means of affinity purification using antibodies or lectins; magnetic beads, adhesion surfaces or elutriation devices. In addition, HLA-matched dendritic cells from a donor can be used and included in the composition.

In a further embodiment, the binding agent-tumor cell complex can be contacted with a dendritic cell ex vivo, which processes the complex by receptor mediated endocytosis, and the dendritic cell preparation can be administered to the patient.

In the embodiments of the invention where the dendritic cell, when added to the composition, is either an immature dendritic cell or is a precursor dendritic cell, the composition is preferably incubated ex vivo under conditions (e.g., in cell culture) such that the immature or precursor dendritic cell matures prior to administering the composition to the patient. Such conditions that allow the formation of mature dendritic cells from immature or precursor dendritic cells are well known to those skilled in the art and are described, for example, in published PCT application WO 01/85204 by Schultes et al.

Accordingly, in one non-limiting method, apoptotic NIH: OVCAR-3 cells and Alt-2 are contacted ex vivo. In a variation of the composition, human anti-murine antibodies are added to the mixture. Subsequently, the mixture is added to immature dendritic cells isolated from a sample from the patient suffering from the disease. The addition of the complex or of a cytokine mixture to apoptotic tumor cells promotes maturation of the immature dendritic cells. Next, the matured dendritic cells “loaded” or “armed” with tumor cells and Alt-2 are removed from culture, optionally purified, and administered to the patient with a binding agent of the present invention. The dendritic cell used in the invention is preferably autologous to the patient to whom the composition of the invention is administered.

One aspect of the present invention includes compositions formulated in pharmaceutically acceptable carriers which can be administered to a patient. On exemplary pharmaceutically acceptable carrier is physiological saline. Other pharmaceutically-acceptable carriers and their formulations are well-known and generally described in, for example, Remington's Pharmaceutical Sciences (18^{th} Edition, ed. A. Gennaro, Mack Publishing Co., Easton, Pa., 1990). In a further embodiment, the pharmaceutical preparations (e.g., compositions) are free from pyrogens.

Another aspect of the present invention is the use of the binding agent in the preparation of a medicament for the treatment of patients suffering from cancer wherein an effective T cell response is elicited in response to the administration of the medicament.

Binding agents of the present invention are unique in that they are effective at low doses of administration. Specifically, the binding agents of the present invention can be administered at a dose of less than or equal to 2 mg per patient and elicit a therapeutic benefit. In a further embodiment, the binding agent is administered to a patient at from about 100 μg to about 2 mg per patient In a further embodiment, the binding agent is formulated in an amount of from about 0.1 μg to about 200 μg per kg of body weight. Binding agents of the present invention can be formulated, for example, for intravenous, intraperitoneal, or subcutaneous administration.

Binding agents of the present invention are capable of inducing a host anti-xenotypic antibody (HAXA) response. In one embodiment, the binding agent is administered at a dosage that elicits a HAXA response of >200 U/ml. In one embodiment, the binding agent is administered at a dosage that elicits a HAXA response of >2000 U/ml. In a further embodiment, the binding agents are capable of inducing a host anti-mouse antibody (HAMA) response. In one embodiment of the present invention, the binding agent is administered at a dosage that is the maximum amount of binding agent that does not induce antibody-mediated toxicity. In a further embodiment, the binding agent is administered at a dosage that is the maximum amount of binding agent that does not produce ADCC or CDC.

In one embodiment of the present invention, the binding agent is conjugated to an immunogenic carrier. In a further embodiment, the immunogenic carrier is keyhole-limpet hemocyanin.

In one embodiment of the present invention, the binding agent is formulated in the presence of an adjuvant to boost the immune system. Adjuvants acceptable for administration to human patients are well-known in the art

In one embodiment of the present invention, the binding agent is formulated in the absence of an adjuvant. In such a formulation, a xenogenic antibody acts as both the binding agent and an adjuvant because it is foreign to the recipient.

One embodiment of the present invention provides for binding agents that cross-link receptors. Binding agents of the invention induce cross-linking of cell-surface receptors via receptor ligation. For example, tumor cells are treated by using antibodies or ligands to receptors that trigger induction of apoptosis such as the receptors of the EGF receptor family or CD20. In a preferred embodiment, the composition contains at least one tumor cell, more preferably the tumor cells are in a concentration of 10⁵ to 10⁸ per patient per treatment. Further, a “ligand-binding site” of a receptor is defmed herein the site on the receptor to which the natural ligand of the receptor binds. For example, if the receptor is a Fcγ type II receptor, the natural ligand for the receptor is an IgG antibody. A binding agent of the invention, when bound to a receptor, blocks the ligand binding site of the receptor such that the natural ligand for that receptor cannot bind the receptor. In one non-limiting example, if the receptor is a Fcγ type II receptor and the binding agent of the invention is an IgG antibody, then binding of the binding agent of the invention to the receptor prevents other IgG antibodies from binding to the receptor.

Pharmaceutical formulations of the present invention can also include veterinary compositions, e.g., pharmaceutical preparations of the binding agents, binding agent-tumor cell complexes, binding agent-tumor cell antigens, dendritic cells suitable for veterinary uses, e.g., for the treatment of livestock or domestic animals, e.g., dogs.

These compounds may be administered to humans and other animals for therapy by any suitable route of administration, including injection (e.g., intravenously, subcutaneously, intradermally, and intraperitoneally).

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

Chemotherapeutic Agents

Chemotherapeutic agents of the invention include chemotherapeutic drugs commercially available.

Merely to illustrate, the chemotherapeutic can be an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and/or a DNA repair inhibitor.

Chemotherapeutic agents may be categorized by their mechanism of action into, for example, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, antlracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphainide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparargine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelanine and thiotepa), alkyl sulfonates-busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGE) inhibitors); angiotensin receptor blocker; nitric oxide donors; anti-sense oligonucleotides; antibodies (trastuzumab, rituximab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors. Preferred dosages of the chemotherapeutic agents are consistent with currently prescribed dosages.

C. Methods of Treatment

One embodiment of the present invention is a method of treating a patient suffering from cancer comprising administering pharmaceutical composition containing a binding agent preparation to the patient whereby the binding agent elicits an effective immune response in the patient, and said effective immune response being categorized as a B and/or T cell response, and whereby the patient receives a therapeutic benefit. An effective B cell response of the present invention can be an effective antibody response. An effective T cell response of the present invention can be an effective T helper response, an effective CTL response, or an effective T helper and CTL response.

In one non-limiting example, a patient suffering from a highly metastatic cancer (e.g., breast cancer) is treated where additional metastasis either does not occur, or are reduced in number as compared to a patient who does not receive treatment. In another non-limiting example, a patient is treated where the patient's solid cancer either becomes partially or totally reduced in size or does not increase in size compared to a patient who does not receive treatment. In yet another non-limiting example, the number of cancer cells (e.g., leukemia cells) in a treated patient is static, or partially or totally reduced compared to the number of cancer cells in a patient who does not receive treatment.

In one embodiment, the patient is a human. In another embodiment, the patient is a non-human mammal, particularly a laboratory animal. Preferred non-human patients of the invention include, without limitation, mice, rats, rabbits, non-human primates (e.g., chimpanzees, baboons, rhesus monkeys), dogs, cats, pigs, and armadillos.

In a further embodiment, the method comprises removing a sample from the patient having either intact tumor cells, or apoptotic tumor cells, or tumor cell antigens, adding an binding agent preparation (e.g., composition) to the sample wherein the binding agent is immunoreactive with a tumor cell antigen present in the sample, allowing a complex to form between the binding agent and tumor cell antigen ex vivo thereby forming a complex, and administering the complex to the patient whereby the patient receives a therapeutic benefit.

In a further embodiment, the binding agent-tumor cell antigen complex is purified prior to administering the complex to the patient. Alternatively, if a tumor cell from a patient sample is not apoptotic, apoptosis-inducing agents can be added to the tumor cells inducing apoptosis prior to mixing in the binding agent preparation.

One aspect of the present invention provides for isolating immature or precursor dendritic cells from a sample taken from a patient. Thus, the immature or precursor dendritic cells of the present invention are autologous to the patient. Additionally, intact tumor cells or, apoptotic tumor cells, or tumor cell antigens are obtained from a sample of the same patient and contacted with a binding agent, thereby forming a complex. The complex is subsequently contacted with the isolated immature or precursor dendritic cells ex vivo such that the dendritic cells process the complex by, for example, receptor-mediated endocytosis and mature. The prepared dendritic cells are then co-administered to the patient with a pharmaceutical composition comprising a binding agent wherein the co-administration elicits an effective immune response in the patient categorized as a T cell response as described above.

In a preferred embodiment of the invention the binding agent and apoptotic tumor cell is targeted in vivo to dendritic cells (which are preferably immature dendritic cells). Such binding occurs through interaction with dendritic cell receptors on the surface of these dendritic cells. By targeting the apoptotic tumor cell to preferably immature dendritic cells and presentation of these tumor cells on both MHC class I and class II molecules, the immune complex of the dendritic cell binding agent/tumor cell efficiently sensitize dendritic cells to induce activation of both CD4(+) helper and CD8(+) cytotoxic T cells in vivo.

A binding agent of the invention may bind to the ligand binding site of a receptor on the surface of a dendritic cell, at any stage of development of the dendritic cell wherein the active portion of the antibody includes a receptor binding site that binds a receptor on dendritic cells with its ligand binding site. Thus, the binding agent includes the Fc portion of an antibody including the heavy chain constant region or the carbohydrate chain at the hinge region. Preferably, once the binding agent is bound to the ligand-binding site of the dendritic cell receptor, the natural ligand cannot bind to the receptor at the same time that the binding agent binds to the receptor. Preferably, the binding agent binds to the receptor on the surface of a dendritic cell when the binding agent is specifically bound to an apoptotic tumor cell. Preferably, such binding causes internalization of the binding agent/apoptotic tumor cell complex. Even more preferably, binding and/or internalization of the binding agent-apoptotic tumor cell complex by an immature or precursor dendritic cell causes maturation and/or activation of the dendritic cell. In a preferred embodiment, the binding agent binds the dendritic cell through the mannose receptor or other C-type lectin. In a preferred embodiment, the binding agent binds the dendritic cell through a complement receptor. More preferably, the binding agent of the invention binds to an activating Fcγ receptor, such as CD64 (FcγRI) or CD32 (FcγRIIA) that is not abundant on neutrophils. Binding agents of the invention are readily identified by art-recognized methods. In one non-limiting example, where the binding agent is an IgG antibody, a precursor, immature, or mature dendritic cell is purified by art known methods and described, for example, in WO 01/85204 by Schultes et al. Subsequently, the dendritic cell is incubated with the FITC labeled IgG antibody (with or without tumor cell to which the antibody specifically binds). Simultaneously or subsequently, a phycoerythrin (PE)-labeled antibody specific for a dendritic cell surface marker is added to the cell. The cell can then be subjected to analysis by flow cytometry to determine if the FITC-labeled IgG antibody of the invention is able to bind to the dendritic cell. The bound receptor can be identified by art-recognized methods. In one non-limiting example, where the binding agent is an IgG antibody, a precursor, immature, or mature dendritic cell is purified by art known methods and described, for example, in WO 01/85204 by Schultes et al. Subsequently, the dendritic cell is incubated with a IgG antibody of the invention (with or without tumor cell to which the antibody specifically binds). Simultaneously, a FITC or the phycoerythrin (PE)-labeled natural ligand or an antibody specific for the ligand binding site of a receptor (i.e., another IgG antibody) is added to the cell. The cell can then be subjected to analysis by flow cytometry to determine if the FITC-labeled IgG antibody of the invention is able to block binding of the PE-labeled receptor ligand or antibody to the receptor on the dendritic cell.

In certain preferred embodiments, the binding agent of the invention is bispecific and binds to both the tumor cell and an Fcγ Type II or Type I receptor on the dendritic cells. Preferably, binding of the binding agent to the Fcγ Type II or Type I receptor blocks the binding of the natural ligand to respectively, the Fcγ Type II or Type I receptor. Accordingly, in certain embodiments, the binding agent binds to the tumor cell and to an Fcγ type I (CD64) receptor on a dendritic cell in the patient administered with the composition. In certain embodiments, the binding agent binds to the antigen and to an Fcγ Type II (CD32) receptor, such as an Fcγ Type IIA (CD32A) receptor on a dendritic cell in the patient administered with the composition. In certain embodiments, the binding agent binds to the tumor cell and to an Fcγ Type III CD16 (FcγRIII) receptor on a dendritic cell in the patient administered with the composition.

In one aspect of the present invention, the method includes the induction of an effective immune response wherein a T cell response is elicited, wherein the T cell response is a T helper response, a CTL response, or both a T helper and a CTL response. In certain embodiments of the methods according to the invention, a CD8+ IFN-γ producing T cell is activated to induce a cytotoxic T lymphocyte (CTL) immune response in the patient administered the composition. In certain embodiments of the methods according to the invention, a CD4+ IFN-γ producing T cell is activated to induce a helper T cell immune response in the patient administered with the composition. These activated CD4+IFN-γ producing T cells (i.e., helper T cells) provide necessary immunological help (e.g. by release of cytokines) to induce and maintain not only CTL, but also a humoral immune response mediated by B cells. Thus, in certain embodiments of the methods according to the invention, a humoral response to the tumor cell is activated in the patient administered with the composition. Activation of a CD8+ and/or CD4+ IFN-γ producing T cells means causing T cells that have the ability to produce IFN-γ to actually produce IFN-γ, or to increase their production of IFN-γ. In preferred embodiments the T cell response is specific for a second distinct antigen present on the tumor cell. In certain embodiments of the methods according to the invention, the T cell response is a T helper response and a CTL response.

In preferred embodiments, the method further comprises administering a chemotherapeutic agent before the composition has been administered to the patient, whereby the chemotherapeutic agent has induced apoptosis resulting in apoptotic tumor cells as defined previously. Thus, patients having already received chemotherapeutic treatment are candidates of the invention. Preferably, the apoptotic tumor cells are circulating within the patient's body. In preferred embodiments the composition is administered within seven days after the chemotherapeutic agent.

In preferred embodiments, the binding agent composition is administered to the patient before a chemotherapeutic agent has been administered to the patient, whereby the chemotherapeutic agent induces apoptosis resulting in apoptotic tumor cells opsonized with the binding agent as described above. Preferably, the apoptotic tumor cell-binding agent complexes are circulating within the patient's body.

In one aspect of the invention, the tumor cell extracted from the patient is exposed to an apoptotic-inducing agent ex vivo, thereby causing the tumor cell to undergo apoptosis. The apoptotic tumor cell is then contacted with the binding agent, thereby forming a complex which can be administered to the patient.

In one aspect of the invention, the method encompasses apoptosis-inducing agents, such as chemotherapeutic agents, radiation, and receptor cross-linking agents. In a preferred embodiment, the apoptosis-inducing agent is a chemotherapeutic agent. Chemotherapeutic agents are well known in the art as described above, and include, for example, genistein and cisplatin. In a preferred embodiment, the apoptosis-inducing agent is radiation. Radiation agents include, for example, gamma radiation. In a preferred embodiment, the apoptosis-inducing agent is cross-linking agent.

In a further embodiment, the antibody-tumor cell complex can be purified prior to administration to the patient such that the complexes are enriched. Purification methods are well-known in the art, and include, for example, affinity purification, cleavage of enterokinase cleavage tags, His-tag sequences, and magnetic bead separation systems.

In one aspect of the present invention, the method includes an additional step of administering a therapeutically acceptable adjuvant to a patient suffering from cancer. The adjuvant can be formulated with the antibody or the complex for administration, or separately.

In one aspect of the present invention, samples can be obtained from patients and include for example, biopsy tissue, blood, or body fluids. Intact tumor cells, apoptotic tumor cells, tumor cell antigens, and dendritic cells can be isolated from the samples using techniques well-known in the art.

In one aspect of the present invention, the patient is administered a chemotherapeutic agent concomitantly with the binding agent-tumor cell antigen complex.

In other aspects of the invention, the tumor cell antigen is present on the surface of an intact tumor cell or apoptotic tumor cell, or is circulating in the blood or body fluid of the patient.

In one embodiment of the present invention, the antibody used to treat the patient having a tumor burden is a xenotypic antibody. In a preferred embodiment, the antibody is a xenotypic monoclonal antibody, or even more preferred, a murine monoclonal antibody. Specific examples of preferred murine monoclonal antibodies include Alt-1, Alt-2, Alt-3, Alt-4, and Alt-5.

Methods of the present invention encompass administration of binding agents, which are therapeutically effective when administered at low doses. Specifically, the binding agents of the present invention can be administered at a dose of less than or equal to 2 mg per patient and exhibit a therapeutic benefit. In a further embodiment, the binding agent is administered to a patient at from about 100 μg to about 2 mg per patient. In a further embodiment, the binding agent is formulated in an amount of from about 0.1 μg to about 200 μg per kg of body weight. Binding agents of the present invention can be formulated, for example, for intravenous, intraperitoneal or subcutaneous administration to a patient suffering from cancer.

When administered to a patient, binding agents of the present invention are capable of inducing a host anti-xenotypic antibody (HAXA) response. In one embodiment of the methods, the binding agent is administered at a dosage that elicits a HAXA response of >200 ng/ml. In one embodiment, the binding agent is administered at a dosage that elicits a HAXA response of >5000 ng/ml. In a further embodiment of the methods, the binding agents induce a host anti-mouse antibody (HAMA) response. In one embodiment of the present invention, the binding agent is administered at a dosage that is the maximum amount of binding agent that does not induce antibody-mediated toxicity. In a further embodiment, the binding agent is administered at a dosage that is the maximum amount of binding agent that does not produce antibody dependent cellular cytotoxicity (ADCC) or complement-dependent cytotoxicity (CDC).

In one embodiment of the present invention, the binding agent is conjugated to an immunogenic carrier prior to administration to a patient. In a further embodiment, the immunogenic carrier is keyhole-limpet hemocyanin.

In one embodiment of the present invention, the binding agent is formulated in the presence of an adjuvant to boost the immune system when administered to a patient. Adjuvants acceptable for administration to human patients are well-known in the art and include, but are not limited to, oligonucleotides, cytokines, alum, or saponins.

In one embodiment of the present invention, the binding agent is formulated in the absence of an adjuvant when administered to a patient. In such a formulation, a xenogenic antibody, for example, acts as its own adjuvant because it is foreign to the recipient.

In one embodiment of the present invention, the patient in need of treatment is suffering from cancer of the prostate, ovaries, breast, stomach, lung, colon, and skin.

In one embodiment of the present invention, the patient in need of treatment is in remission. In a preferred embodiment, the patient in need of treatment is a human.

D. Pharmaceutical Packages

One embodiment of the present invention is a pharmaceutical package comprising a pharmaceutical composition comprising a binding agent, or fragment thereof, that is immunoreactive with a tumor cell antigen on an intact tumor cell or an apoptotic tumor cell, or with a circulating tumor cell antigen and instructions for the administration to a patient suffering from cancer. In the following embodiments, the term “tumor cell antigen” is meant to be interchangeable with tumor cell antigen on an intact tumor cell or an apoptotic tumor cell, and circulating tumor cell antigen which may or may not be circulating in body fluids.

In a preferred embodiment, the binding agent is an antibody, or fragment thereof. The antibody can administered to a patient and bind to a tumor cell antigen on the surface of an apoptotic tumor cell or a tumor cell that is subsequently induced to undergo apoptosis in vivo. Alternatively, a sample containing a tumor cell antigen or a tumor cell can be taken from the patient, reacted with the antibody ex vivo, thereby forming an antibody-tumor cell antigen complex. The tumor cell is either apoptotic before combined with the binding agent or is induced to undergo apoptosis after the binding agent is bound. The complex can then be administered to the patient for the treatment of cancer. Additionally, the antibody-tumor cell antigen complex can be purified/enriched such that the concentration of complexes administered to the patient are increased.

The pharmaceutical package of the present invention may additionally contain an apoptosis-inducing agent, wherein the apoptosis-inducing agent is, for example, a chemotherapeutic agent, radiation, or a receptor cross-linking agent. Chemotherapeutic agents, radiation, and receptor cross-linking agents have been discussed above. Exemplary chemotherapeutic agents include, for example, genistein and cisplatin.

In a further embodiment, the antibody can be administered to a patient either alone, or co-administered with an apoptosis-inducing agent, thereby eliciting an effective B and/or T cell response. The T cell response elicited can be a T helper response, a CTL response, or a T helper and CTL response.

The pharmaceutical package of the instant invention may also contain an adjuvant to be administered to the patient whereby the B and/or T cell response elicited by the antibody and/or apoptosis inducing agent is enhanced.

In an alternative embodiment, the antibody composition of the pharmaceutical package can be administered about a week prior to administration of an apoptosis-inducing agent. Alternatively, the antibody can additionally be administered as needed after the apoptosis-inducing agent to enhance the B and/or T cell response elicited.

The compositions of the pharmaceutical package of the present invention can be formulated in single or multiple dose volumes such that the compositions can be administered to a patient as needed in order to elicit a therapeutically beneficial B and/or T cell response.

In a preferred embodiment of the present invention, the antibody composition of the pharmaceutical package is a xenotypic antibody. In a further invention, the xenotypic antibody is a xenotypic monoclonal antibody. Specific examples of antibodies include, for example, Alt-1, Alt-2, Alt-3, Alt-4, and Alt-5.

In a preferred embodiment of the present invention, the pharmaceutical package additionally comprises HLA-matched dendritic cells that are autologous to the patient to be treated.

Alternatively, in a preferred embodiment of the present invention, the pharmaceutical package additionally comprises antibodies that can be used to isolate dendritic cells from a patient. Such antibodies can be obtained, for example, from Pharmingen (San Diego, Calif.).

Alternatively, in a preferred embodiment of the present invention, the pharmaceutical package additionally comprises a cassette that can be used to isolate immature DC from a patient, culture the cells ex vivo, and isolate the cells such that they can be combined with the antibody and tumor cell prior to re-administration of the matured dendritic cells to the patient. Such cassettes can be obtained, for example, from Aastrom's Biosciences, Inc.

In preferred embodiments, the compositions of the pharmaceutical package are approved for treatment of human patients and are free of pyrogens.

E. Administration

These materials may be administered orally; or by intravenous injection; or by injection directly into an affected tissue, as for example by injection into a tumor site, or intraperitoneally, intradermally, or subcutaneously.

Compositions of the present invention are administered in a therapeutically effective amount such that an effective immune response as described above is elicited.

F. Exemplary Tumors for Treatment

Antibodies of the present invention inhibit the proliferation of or induce apoptosis of: a pancreatic tumor cell, a lung tumor cell, a prostate tumor cell, a breast tumor cell, a colon tumor cell, a liver tumor cell, a brain tumor cell, a kidney tumor cell, a skin tumor cell and an ovarian tumor cell, and therefore inhibit the growth of a squamous cell carcinoma, a non-squamous cell carcinoma, a glioblastoma, a sarcoma, an adenocarcinoma, a melanoma, a papilloma, a neuroblastoma and a leukemia cell.

The method of present invention is effective in treatment of various types of cancers, including but not limited to: pancreatic cancer, renal cell cancer, Kaposi's sarcoma, chronic leukemia, breast cancer, sarcoma, ovarian carcinoma, rectal cancer, throat cancer, melanoma, colon cancer, bladder cancer, mastocytoma, lung cancer, mammary adenocareinoma, pharyngeal squamous cell carcinoma, gastrointestinal cancer, stomach cancer, or prostate cancer.

### IV. Equivalents

Those skilled in the art will recognize, or be able to ascertain using no more than routine experimentation, many equivalents to the specific embodiments of the invention described herein. Such equivalents are intended to be encompassed by the following claims.

All of the above-cited references and publications are hereby incorporated by reference in their entireties.

The following examples are intended to further illustrate certain particularly preferred embodiments of the invention and are not intended to limit the scope of the invention.

### EXAMPLE I

Materials and Methods

Materials

The murine monoclonal anti-CA125 antibody B43.13 (AltaRex Corporation, Edmonton, Alberta, Canada) was produced in mouse ascites and purified by Protein A affinity and anion exchange chromatography. This IgG1 antibody reacts specifically and with high affinity with CA125. Chemotherapeutic agents (paclitaxel, doxorubicin, topotecan, carboplatin) were obtained from LKT Labs.

Cells and Source of Cells

NIH:OVCAR-3 ovarian cancer cell line was purchased from ATCC (Manassas, Va.). Peripheral Blood Leukocytes (PBL) of healthy normal donors were obtained by leukaphoresis (SeraCare, Calif.) and purified on a Histopaque gradient (Sigma, Mississauga, Canada), viably frozen in 90% human Ab serum (Gemini Bio-Products, Woodland, Calif.) and 10% DMSO (Sigma, St. Louis, Mo.) and stored in the vapor phase of liquid nitrogen until used. DNA was prepared from a portion of the cells and used for molecular HLA typing.

Source of Cells

PBMC were isolated from the apheresis products from normal volunteers by ficoll-hypaque (Sigma, St. Louis, Mo.) gradient centrifugation, viably frozen in 90% human Ab serum (Gemini Bio-Products, Woodland, Calif.) and 10% DMSO (Sigina, St. Louis, Mo.) and stored in the vapor phase of liquid nitrogen until used. DNA was prepared from a portion of the cells and used for molecular HLA typing.

Isolation of DC by Negative Selection

DC precursors were prepared from freshly-thawed PBMC by negative selection using immunomagnetic bead depletion of lineage cells. PBMC were incubated on ice for 30 min with mouse anti-human CD3, CD16 and CD19. Excess antibody was removed by washing the cells with PBS/0.1% BSA and the cells were incubated with Pan Mouse IgG immunomagnetic beads for 30 min on ice (Monocyte isolation kit, Dynal, Lake Success, N.Y.). The tube was placed against a magnet to remove the cell:bead complexes and the supernatant containing the lineage-depleted DC precursors collected.

DC Cultures

The lineage-depleted DC precursors were washed, resuspended in cRPMI (RPMI supplemented with 1% glutamine and 10% heat-inactivated human Ab serum) containing GM-CSF (1000 U/ml) and IL-4 (1000 U/ml)(R & D Systems, Minneapolis, Minn.) and cultured at 37° C. in 5% CO₂ at 0.5×10⁶ cells/well in 24 well plates. On the fourth day of culture, the cells were pulsed with antigen and incubated for an additional 8-24 h. TNFα (10 μg/ml) and IFNα (50 μg/ml), known to mature DC, were then added to the cultures. The matured DC were harvested on the seventh day of culture, analyzed for phenotypic markers by flow cytometry and used in functional studies.

Phenotypic Analysis of DC by Flow Cytometry

DC were analyzed for cell surface marker expression by flow cytometry. Briefly, the cells were aliquoted into polystyrene tubes and stained for surface markers with fluorochrome-labeled murine antibodies. Cell surface markers include: HLA-A,B,C, HLA-DR, CD14, CD11c, CD4, CD40, CD83, CD86, CD80, CD16, CD32, CD64 (Becton Dickinson, San Jose, Calif.). Following a 30 min incubation on ice, the cells were washed with PBS and pelleted by centrifugation. The cell pellets were resuspended in 250 μl of fixative (2% paraformaldehyde). The data was acquired using a FACScan flow cytometer (Becton Dickinson, San Jose, Calif.) and analyzed with Cellquest software (Becton-Dickinson, San Jose, Calif.).

Isolation of T Cells

Responder CD3+ T lymphocytes were isolated from thawed PBMC by negative selection (T cell isolation kit, Dynal, Lake Success, N.Y.). Briefly, the cells were incubated on ice for 30 min with a mixture of antibodies to CD14, CD16, CD56 and HLA Class II DR/DP. Excess antibodies were removed by washing with PBS/0.1% BSA. The cells were incubated for 30 min at room temperature with immunomagnetic beads coated with an anti-mouse IgG antibody. The cells were placed against a magnet and the T lymphocytes were isolated from the supernatant

Preparation of Tumor Cells

The murine monoclonal anti-CA125 antibody B43.13 (AltaRex Corporation, Edmonton, Alberta, Canada) was produced in mouse ascites and purified by Protein A affinity and anion exchange chromatography. This IgG1 antibody reacts specifically and with high affinity with CA125. NIH:OVCAR-3 tumor cells were rendered apoptotic by gamma irradiation (10,000 rad) or by chemotherapeutic agents. Chemotherapeutic agents were incubated with the tumor cells at the IC₉₀ (concentration required to induce 90% cell killing) for 4-24 h, followed by washing of the cells). Tumor cells and B43.13 were diluted in cRPMI to concentrations of 500 U/mL, 5,000 cells/mL and 5 μg/ml, respectively, and loaded into the dendritic cells.

In vitro Activation of T Cells

NIH:OVCAR-3 tumor cells were induced to undergo apoptosis by irradiation (10,000 Rad), or with chemotherapeutic drugs (4-24 h incubation), washed, and fed to BLA-matched immature DC. In parallel, a set of apoptotic cells were incubated with MAb-B43.13 prior to loading of immature DC. As a control, necrotic NIH:OVCAR-3 cells (repeated freeze-thaw cycles) were fed to immature DC with and without MAb-B43.13. DC were loaded for 2 h at a ratio of tumor cells per DC, matured and incubated for 3 days. On day 7, DC were harvested and washed, and purified autologous T cells were added at a ratio of 10:1 (T cells to DC) and cultured for another 7 days. At day 7 the T cells were harvested, washed and cultured for an additional 7 days with DC that had been armed as described above in cRPMI supplemented with IL-2 (10 U/ml) and IL-7 (5 ng/ml)(R&D Systems, Minneapolis, Minn.) at a ratio of 20:1. T cells were restimulated for 24 h with armed DC (in combinations described in the Results) and responses assessed by measuring intracellular cytokine production in CD4+ and CD8+ T lymphocytes or in chromium release assays against NIH:OVCAR-3 cells.

Chromium Release Assay

NIH:OVCAR-3 cells were harvested when 50-80% confluent by trypsinisation. Cells were washed and 2×10⁶ cells were resuspended in 100 μL RPMI+20 μL FBS+2 mCi ⁵¹Cr. Cells were incubated for 2 h at 37° C. to allow for incorporation of chromium, then cells were washed and plated into round-bottom microtiter plates at 10⁴ cells/well/100 μL. T cell cultures 2 h after restimulation with antigen armed DC were added to the labeled cells at effector to target cell ratios of 20:1 to 0.625:1 (100 μL/well) and, as controls, 100 μL of medium (spontaneous release) or 0.1% Tween-20 (maximum release) were added. Plates were incubated for 4 h at 37° C. and then centrifuged at 30×g for 5 min. One hundred μL aliquots of the supernatants were collected and counted in a gamma counter. Specific lysis was calculated according to the formula: % specific release=(dpm obtained with specific sample−dpm for spontaneous release)/(dpm for Maximum release−dpm for spontaneous release)×100.

WST-1 for Monitoring Drug-Induced Cell Death

NIH:OVCAR-3 cells were grown in 96-well plates (NUNC) and irradiated with 10,000 rad or treated with chemotherapeutic drugs in a range of concentrations for 4 h, followed by washing. Cells were incubated at 37° C. for up to 3 days. WST-1 substrate (Boehringer-Mannheim, Mannheim, Germany) was added for 4 h 24, 48, and 72 h after treatment. Plates were read in an ELISA reader at 650 nm and the percentage of cell death calculated according to the formula: A650 of treated cells/A650 of untreated cells×100.

Annexin V Apoptosis Assay

NIH:OVCAR-3 cells were grown in 6-well plates (NUNC) and irradiated or treated with chemotherapeutic drugs for 4-48 h, washed and stained with Annexin V-FITC (BD-Pharmingen) for 1 h. Cells were analyzed by flow cytometry (Becton-Dickinson, CellQuest), counterstained with Propidium Iodide and analyzed again in the flow cytometer.

Confocal Microscopy for Activated Caspases

NIH:OVCAR-3 cells were grown in tissue chamber slides (NUNC) and treated with chemotherapeutic drugs for 4-48 h, washed and stained with Phi-Phi-Lux for 1 h. Cells were washed briefly again, fixed and counterstained with Propidium Iodide prior to analysis by confocal microscopy (Zeiss, Germany).

CA125 Expression

NIH:OVCAR-34 cells were analyzed for CA125 expression prior to and 24 h after apoptosis induction with chemotherapeutic drugs or irradiation. Cells were incubated on ice with FITC-labeled MAb-B43.13 (FITC labeling kit, Molecular Probes, Eugene Oreg.) at 5 μg/mL for 1 h, washed twice, fixed and analyzed by flow cytometry.

Dendritic Cell Uptake of Tumor Cell by Confocal Microscopy

Immature dendritic cells were grown in chamber slides and incubated for 4 h-72 h with CFSE-labeled tumor cells undergoing apoptosis with and without MAb-B43.13 opsonization. Cells were fixed, permeabilized and stained with DAPI and antibodies against toll-like receptors 2, 3 and 6, followed by anti-rabbit-PE.

Antigen Stimulation Assays

T lymphocytes were plated in twenty-four well plates at a concentration of 1×10⁶ cells/well and to which were added 5×10⁴ DC that were antigen naive or that had been exposed to MAb-B43.13, NIH:OVCAR-3 cells, or NIH:OVCAR-3 cells+MAb-B43.13. At day 7 the T cells were harvested, washed and cultured for an additional 7 days with DC that had been armed as described above in cRPMI supplemented with IL-2 (10 U/ml) and IL-7 (5 ng/ml)(R&) Systems, Minneapolis, Minn.). T cells were restimulated for 24 h with armed DC (in combinations described in the Results) and responses assessed by measuring intracellular cytokine production in CD4+ and CD8+ T lymphocytes or in chromium release assays against NIH:OVCAR-3 cells.

Detection of Intracellular Cytokine Expression by Flow Cytometry

Intracellular cytokine production by CD4+ and CD8+ T cells was measured by flow cytometry. Brefeldin A (10 μg/ml)(Pharmingen, San Diego, Calif.) was added to the T cell cultures 2 h after restimulation with antigen armed DC. After an additional 18 h of culture, cells were incubated with staining buffer (PBS with 1% human Ab serum) for 15 min at 4° C., washed again, pelleted and fluorochrome labeled antibodies to CD3, CD4, or CD8 (Becton Dickinson, San Jose, Calif.) added. The cells were fixed and permeabilized by incubation with perm/fix solution (Pharmingen, San Diego, Calif.) for 20 min on ice, washed and antibodies to IFNγ or appropriate isotype controls (Pharmingen, San Diego, Calif.) added. After incubation for 30 min on ice, the cells were washed, resuspended in staining buffer containing 2% paraformaldehyde and analyzed by flow cytometry.

Chromium Release Assay

NIH:OVCAR-3 cells were harvested when 50-80% confluent by trypsinisation. Cells were washed and 2×10⁶ cells were resuspended in 100 μL RPMI+20 μL FBS+2 mCi⁵¹Cr. Cells were incubated for 2 h at 37° C. to allow for incorporation of chromium, then cells were washed and plated into round-bottom microtiter plates at 10⁴ cells/well/100 μL. T cell cultures 2 h after restimulation with antigen armed DC were added to the labeled cells at effector to target cell ratios of 20:1 to 0.625:1(100 μL/well) and, as controls, 100 μL of medium (spontaneous release) or 0.1% Tween-20 (maximum release) were added. Plates were incubated for 4 h at 37° C. and ten centrifuged at 30×g for 5 min. One hundred μL aliquots of the supernatants were collected and counted in a gamma counter. Specific lysis was calculated according to the formula: % specific release=(dpm obtained with specific sample−dpm for spontaneous release)/(dpm for Maximum release−dpm for spontaneous release)×100.

Results

Induction of Apoptosis by Irradiation and Chemotherapeutic Drugs

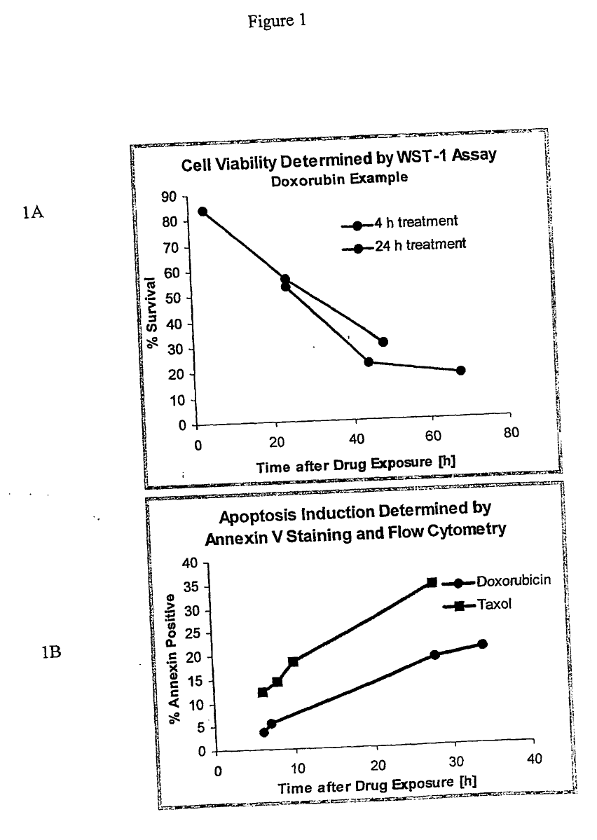

Drug concentrations were optimized using NIH:OVCAR-3 cells and WST-1 assay to achieve 90% cell killing (IC₉₀) within 3 days. The optimum concentrations are described in each example. NIH:OVCAR-3 cells were treated with paclitaxel at 1 μg/mL (IC₉₀) in chamber slides, washed and incubated at 0, 4, 24 and 48 h with the fluorescent caspase 3 substrate Phi-Phi-Lux. Cells were counterstained with Propidium Iodide, washed again and analyzed by confocal microscopy. Paclitaxel-induced apoptosis peaked at 4 h after treatment. At this time point more than 60% of the cells stained positive for activated caspase but only very few cells for PI (cells that have died from the treatment). In contrast, at the 24 h time point about half of the cells were found dead and lesser cells stained positive for caspase activity. By 48 h almost all cells were dying and very few cells showed signs of apoptosis. Similar results were obtained with Annexin V staining and flow cytometry for monitoring of apoptosis and WST-1 assay for assessment of cell death using doxorubicin (FIG. 1) and paclitaxel as well as topotecan, carboplatin and irradiation. Based on these data, a 4 h drug treatment time was chosen for all experiments.

Antigen Expression by Live and Apoptotic Tumor Cells

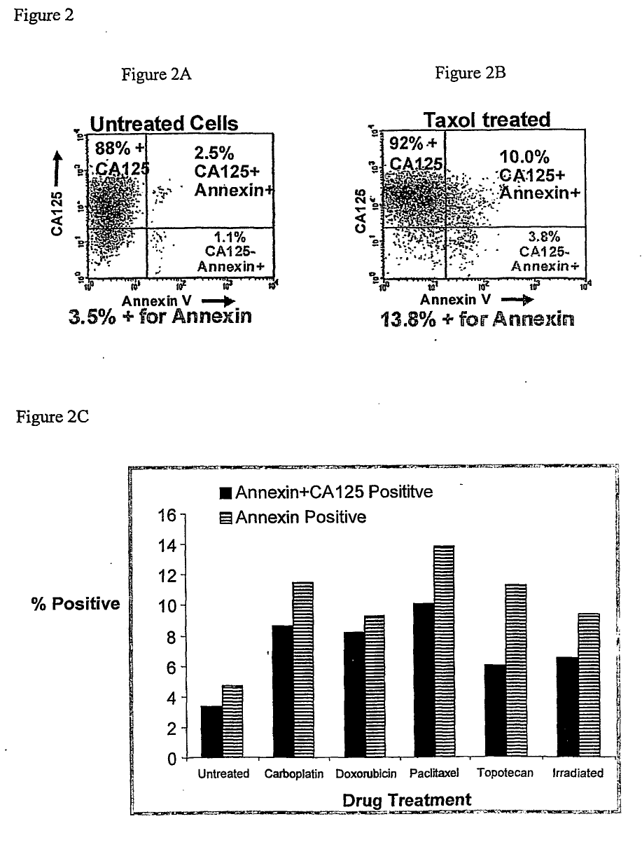

As demonstrated in FIG. 2, apoptotic tumor cells are positive for the targeted tumor-associated antigen CA125. The cells are more than 90% positive for the CA125 antigen and cell undergoing apoptosis (positive staining for Annexin V) are also positive for CA125.

Endocytosis of Tumor Cells

Tumor cells, labeled with the fluorescent dye CFSE were fed to dendritic cells with and without addition of the binding agent MAb-B43.13. Cells were fixed, then dendritic cells were visualized using PE-labeled anti-CD11c (a marker specific for dendritic cells. The cell nuclei were stained with DAPI. Tumor cells that are opsonized with the binding agent MAb-B43.13 are endocytosed by dendritic cells.

Induction of Cytolytic T Cells

NIH:OVCAR-3 tumor cells were rendered apoptotic by irradiation (10,000 rad). Cells were removed from culture dishes by trypsin digestion, centrifuged and resuspended in cRPMI. A portion of the cells was incubated with 5 μg/ml of MAb-B43.13. MAb-B43.13 antibody (5 μg/mL), apoptotic tumor cells with and without MAb-B43.13 (1 tumor cell per DC) or control medium were fed to immature DC, and DC were matured 1 h later. T cells were added on Day 7 (20 T cells per DC), cultured for 7 days and restimulated twice with DC that had been armed as described above. T cell cultures 24 h after final stimulation were added to chromium labeled target cells at effector to target cell ratios of 20:1 to 0.625:1 for 4 h. Supernatants were counted and specific lysis calculated.

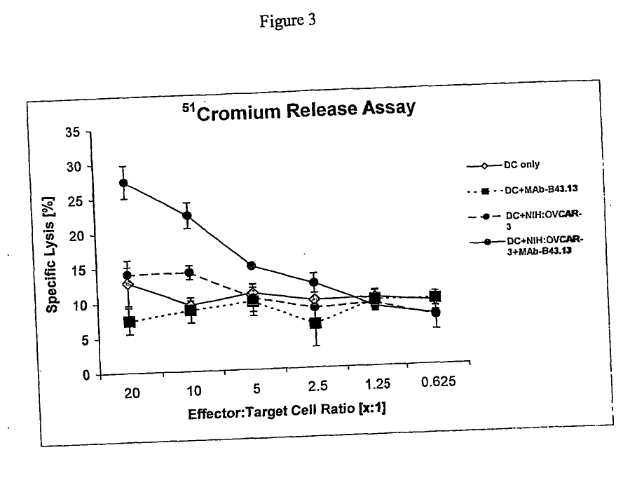

Results demonstrated that the ex vivo administration of dendritic cells, tumor cells, and binding agent were superior in lysing tumor than dendritic cell alone or in combination with binding agent or tumor cell. Results are illustrated in FIG. 3.

### Example II

Twenty human patients diagnosed with recurrent ovarian cancer entered a study of non-radiolabeled murine MAb-B43.13 in combination with standard chemotherapeutic agents. Patients received twenty minute infusions of 2 mg of MAb-B43.13 at weeks 1, 3, 5, and 9, and a further optional dose at week 12. After treatment with MAb-B43.13, patients received standard chemotherapy and an optional dose between weeks 12 and 26. Disease progression was assessed using CT scans, physical exam, CA125 levels, and long-term follow-up for survival. T cell responses to autologous tumor were assessed in eight patients using ELISPOT Assay.

T Cell Responses

Patients peripheral blood mononuclear cells (PBMC) were thawed using standard techniques. The PBMC were allowed to sit for 2 minutes in the DNAse thaw media before washing. PBMC were washed once by first adding 8 mL AIM V media (commercially available from Gibco/Invitrogen Corporation, Carlsbad, Calif.). PBMC were resuspend in 10 mL AIM V media. 3-8×10⁶/mL PBMC in 10 ml AIM-V were incubated for one hour at 37° C., 5% CO₂ in a T75 flask plate.

After the incubation, the flask was washed with warm AIM V media four times (10 mL each wash), by adding the warm media to the side of the flask, not directly onto the adhered cells and decanting after each wash as well as aspirating the final wash.

After the final wash, Isocve's Modified Dulbecco's Media (10 mL IMDM commercially available from Gibco/Invitrogen Corporation, Carlsbad, Calif.), Fetal Bovine Serum (10% FBS commercially available fiom Gibco/Invitrogen Corporation, Carlsbad, Calif.), GM-CSF (1,000 U/ml), and IL-4 (1,000 U/ml) (both commercially available from R&D Systems, Minneapolis, Minn.) were added to the flask and incubated for 3 days at 37° C., 5% CO₂.

On day 3 the dendritic cell culture was fed by adding IMDM (2 mL), FBS (10%), GM-CSF (12,000,U), and IL-4 (12,000,U) to the flask (final cytokine concentration in flask was 1,000 U/ml GM-CSF, and 1,000 U/ml IL-4). Antibody and antigen were then complexed on day 6 for one hour at 37° C., 5% CO₂ in AIM-V. While complex was incubating, dendritic cells were harvested by tapping the flask after incubation with 4° C. PBS for 15 minutes at 4° C. Dendritic cells were then washed in plain AIM-V media (24 mL) and counted. A total of 25,000 to 100,000 dendritic cells were added to a 12 well plate. Antigen/antibody complex was then added to each well and incubated in a total volume of 1 mL for 4 hours at 37° C., 5% CO₂.

Supernatant was removed and AIM-V (1 mL), TNF-α (10 ng/mL), IL-1β (10 ng/mL), and IL-6 (10 ng/mL) (commercially available from R&D Systems) was added to the culture and incubated overnight at 37° C., 5% CO₂.

The following day the in vitro stimulation was initiated by thawing patient T cells obtained from various time points (i.e., 12 weeks sample prior to chemotherapy and 26 week sample post chemotherapy). Cells were counted and resuspended with RPMI-1640 (1-2×10⁶ mL commercially available from Life Technologies, Frederick, Md.), FBS (10%), L-glutamine and gentamycin (commercially available from R&D Systems, Minneapolis, Minn.), IL-2 (20 IU/mL) and IL-7 (10 ng/mL).

Media was aspirated from the cultured dendritic cells and washed with AIM-V media. Patient T cells were then added at a ratio of 10-50:1 and incubated for 10 days at 37° C., 5% CO₂.

On day 10 the culture was fed with RPMI-1640 (0.5 mL), FBS (10%), L-glutamine and gentamycin, and IL-2 (80 IU/mL) and incubated for three days at 37° C, 5% CO₂.

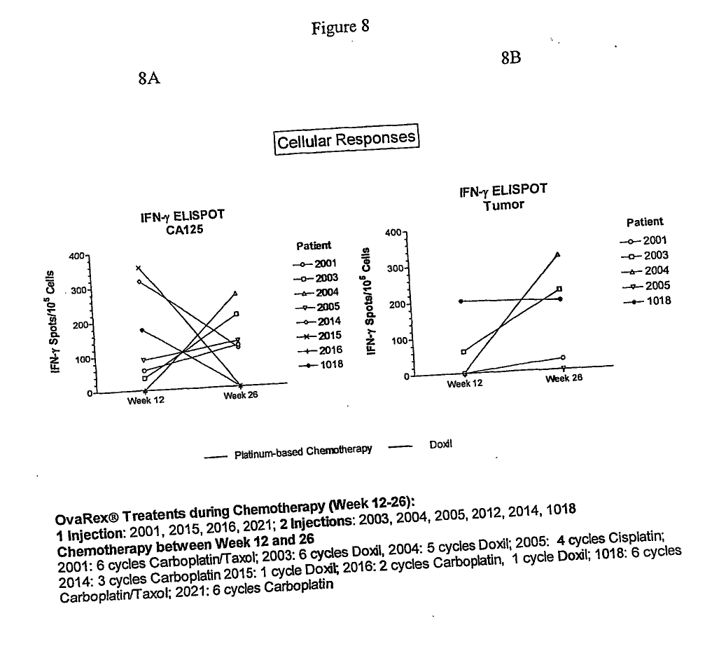

Results were analyzed using ELISPOT assay for T cell secretion of IFN-γ. Patients receiving non-radiolabeled MAb-B43.13 demonstrated tumor-specific T cells post-administration as illustrated in FIG. 8. T cell samples taken at the 8 week time point (MAb-B43.13 administered prior to chemotherapy) had a lower T cell response to autologous tumor than patient samples taken at the 26 week time point (non-radiolabeled MAb-B43.13 administration post chemotherapy) as illustrated in FIG. 8.

Beneficial Treatment Effect of T cell Responses

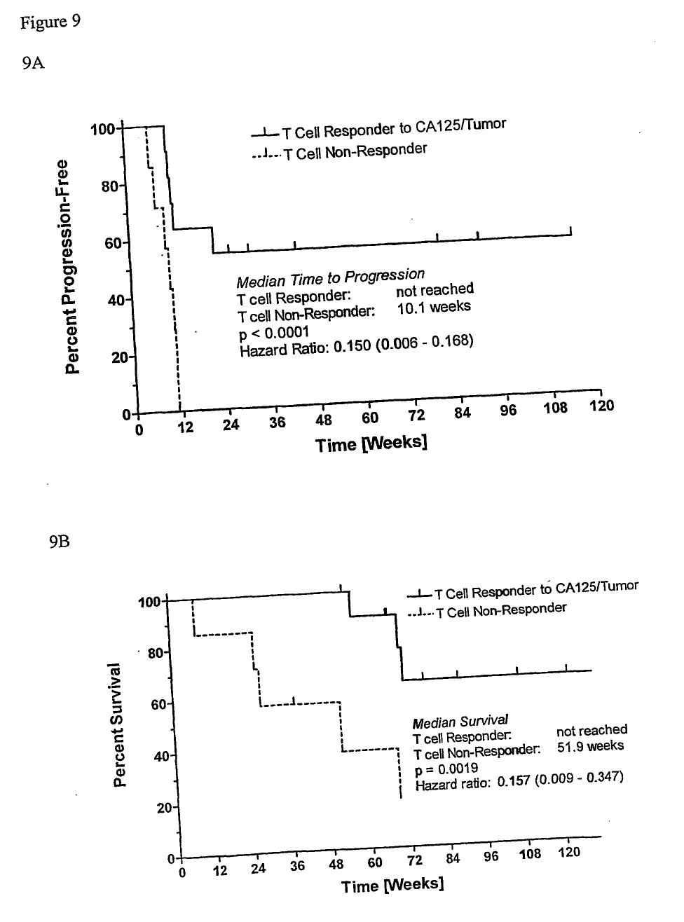

Using statistical analysis, time to progression and survival advantages were correlated with T cell responses to autologous tumor. Patients that exhibited a T cell response to autologous tumor and/or CA125 and had a significant increase in time to progression (60 weeks vs. 10.7 weeks) as illustrated in the Kaplan Meier representation of FIG. 9B. Additionally, patients who exhibited a T cell response to autologous tumor and/or CA125 also had a significant increase in survival (median not reached at the 108 week time point vs. median of 38 weeks) as illustrated in the Kaplan Meier representation of FIG. 9A.

### Example III

Assays were preformed as described for Example I with the following modifications. NIH:OVCAR-3 tumor cells were purchased from ATCC, Manassas, Va. The murine monoclonal anti-CA125 antibody B43.13 (AltaRex Corporation, Edmonton, Alberta, Canada) was produced in mouse ascites and purified by Protein A affinity and anion exchange chromatography. This IgG1 antibody reacts specifically and with high affinity with CA125. NIH:OVCAR-3 tumor cells were rendered apoptotic by treatment with Taxol (1 μg/mL) or doxorubicin (100 μg/mL) for 24 h. Cells were washed and removed from culture dishes by trypsin digestion, centrifuged and resuspended in cRPMI. A portion of the cells was incubated with 5 μg/ml of MAb-B43.13 for 30 minutes on ice and washed again, whereas the remaining cells were incubated on ice for 30 minutes without addition of antibody. NIH:OVCAR-3 cells were also rendered necrotic by submitting them to 3 cycles of freeze-thaw. MAb-B43.13 antibody (5 μ/mL), apoptotic and necrotic tumor cells with and without MAb-B43.13 (1 tumor cell per DC) were fed to immature DC. After a 1 h incubation, DC were matured utilizing maturing agents (TNF-α, 10 ng/mL; and IFN-γ, 50 U/mL) that were added and the cells incubated for another 3 days.

T lymphocytes were added on Day 7 and cultured with loaded DC as described in Example I, plated in twenty-four well plates at a concentration of 1×10⁶ cells/well and to which were added 5×10⁴ mature DC that were antigen naive or that had been exposed to MAb-B43.13, apoptotic or necrotic NIH:OVCAR-3 cells, or apoptotic or necrotic NIH:OVCAR3 cells+MAb-B43.13. At day 7 the T cells were harvested, washed and cultured for an additional 7 days with DC that had been armed as described above in cRPMI supplemented with IL-2 (10 U/ml) and IL-7 (5 ng/ml)(R&D Systems, Minneapolis, Minn.).

T cell cultures 24 h after final re-stimulation with antigen armed DC were added to chromium labeled cells (see Example I) at effector to target cell ratios of 25:1 to 2.5:1 (100 μL/well) for 4 h and as controls, 100 pL of medium (spontaneous release) or 0.1% Tween-20 (maximum release) were added. Plates were incubated for 4 h at 37° C. and ten centrifuged at 30×g for 5 min. One hundred μL aliquots of the supernatants were collected, and counted in a gamma counter, and specific lysis was calculated according to the formula: % specific release=(dpm obtained with specific sample−dpm for spontaneous release)/(dpm for Maximum release−dpm for spontaneous release)×100.

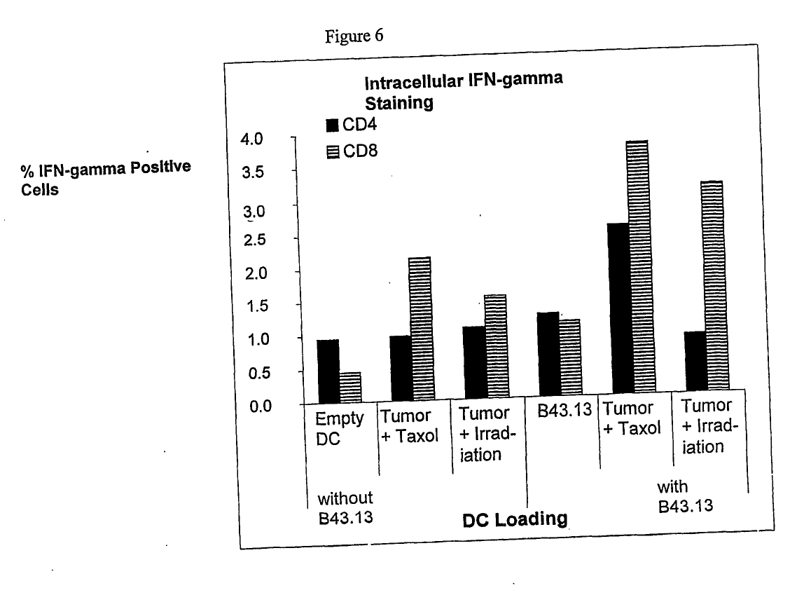

Results demonstrated that the ex vivo administration combination of dendritic cells, Taxol- or doxorubicin-treated apoptotic tumor cells, and binding agent combined together were superior in lysing tumor cells than dendritic cells alone or in combination with binding agent alone, apoptotic tumor cell alone, necrotic tumor cells alone or necrotic tumor cells and binding agent. Results are illustrated in FIG. 6.

### Example IV

Assays were performed as described for Example 1 with the following modifications. NIH:OVCAR-3 tumor cells were purchased from ATCC, Manassas, Va. The murine monoclonal anti-CA125 antibody B43. 13 (AltaRex Corporation, Edmonton, Alberta, Canada) was produced in mouse ascites and purified by Protein A affinity and anion exchange chromatography. This IgG1 antibody reacts specifically and with high affinity with CA125. NIH:OVCAR-3 tumor cells were rendered apoptotic by treatment with the chemotherapeutics doxorubicin (100 μg/mL, Taxol (1 μg/mL), topotecan (2.5 μg/mL) and carboplatin (100 μg/mL) for 24 h or by irradiation (10,000 rad) as well as made necrotic by repeated freeze-thaw. Cells were washed and removed from culture dishes by trypsin digestion, centrifuged and resuspended in cRPMI. A portion of the cells was incubated with 5 μg/ml of MAb-B43.13 for 30 min. on ice and washed again, whereas the remaining cells were incubated on ice for 30 min. without addition of antibody. MAb-B43.13 antibody (5 μg/mL), apoptotic and necrotic tumor cells with and without MAb-B43.13 (1 tumor cell per DC) were fed to immature DC.DC were matured and after a 1 h incubation, maturing agents (TNF-α, 10 ng/mL; and IFN-γ, 50 U/mL) were added and the cells incubated for another 3 days cultured with T cells as described in Examples I and II.

T lymphocytes were plated in twenty-four well plates at a concentration of 1×10⁶ cells/well and to which were added 5×10⁴ mature DC that were antigen naive or that had been exposed to MAb-B43.13, apoptotic or necrotic NIH:OVCAR-3 cells, or apoptotic or necrotic NIH:OVCAR3 cells+MAb-B43.13. At day 7 the T cells were harvested, washed and cultured for an additional 7 days with DC that had been armed as described above in cRPMI supplemented with IL-2 (10 U/ml) and IL-7 (5 ng/ml)(R&D Systems, Minneapolis, Minn.).

T cell cultures 24 h after final re-stimulation with antigen armed DC were added to chromium labeled cells (see Example I) at an effector to target cell ratios of 25:1 to 2.5:1(100 pL/well) and as controls, 100 μL of medium (spontaneous release) or 0.1% Tween-20 (maximum release) were added. Plates were incubated for 4 h at 37° C. and ten centrifuged at 30×g for 5 min. One hundred μL aliquots of the supernatants were collected, and counted in a gamma counter, and s. Specific lysis was calculated according to the formula: % specific release=(dpm obtained with specific sample−dpm for spontaneous release)/(dpm for Maximum release−dpm for spontaneous release)×100.

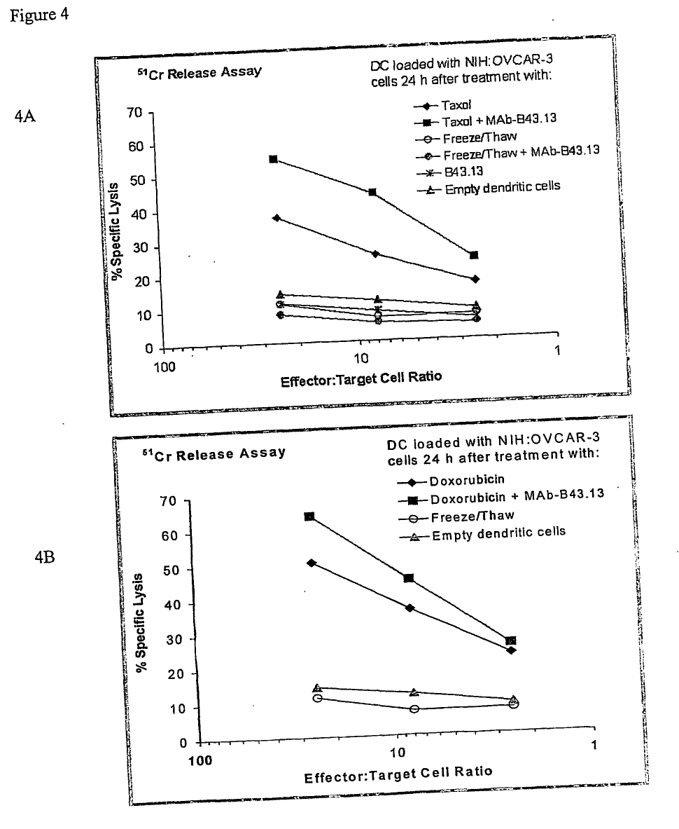

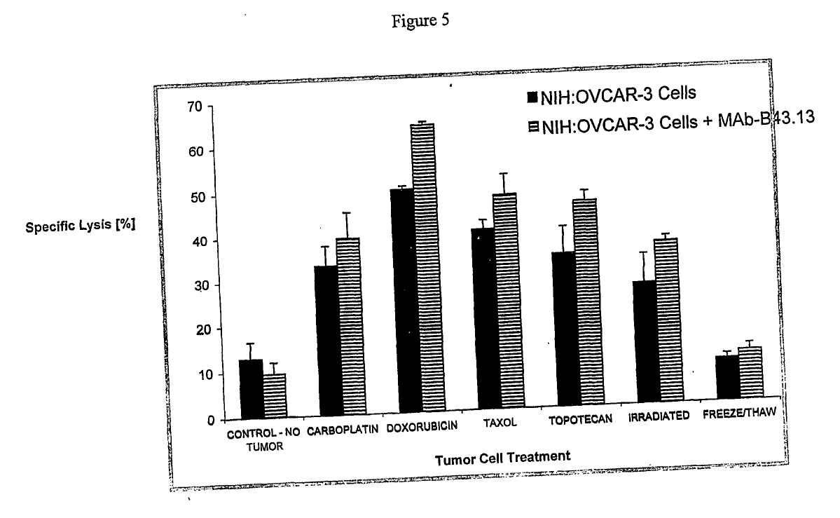

Results demonstrated that the ex vivo administration combination of dendritic cells, doxorubicin-treated apoptotic tumor cells, and binding agent together were superior in lysing tumor than dendritic cell alone or in combination with binding agent alone, apoptotic tumor cell alone or necrotic tumor cell alone or necrotic tumor cell and binding agent. Tumor cells, rendered apoptotic by all four tested chemotherapeutic drugs, were more effective in inducing CTL than tumor cells rendered apoptotic by irradiation. Apoptotic tumor cells coated with the binding agent MAb-B43.13 prior to loading onto DC were more potent activators of CTL than apoptotic tumor cells alone or the binding agent alone for all apoptosis agents tested. Results are illustrated in FIG. 4.

### Example V

Assays were performed as described for Example I with the following modifications. NIH:OVCAR-3 tumor cells were rendered apoptotic by treatment with Taxol (1 μg/mL) for 24 h. Cells were washed and removed from culture dishes by trypsin digestion, centrifuged and resuspended in cRPMI. A portion of the cells was incubated with 5 μg/ml of MAb-B43.13 for 30 minutes on ice and washed again, whereas the remaining cells were incubated on ice for 30 minutes without addition of antibody. MAb-B43.13 antibody (5 μg/mL), apoptotic tumor cells with and without MAb-B43.13 (1 tumor cell per DC) were fed to immature DC. After a 1 h incubation, DC were matured. T lymphocytes were added on Day 7 and cultured with loaded DC as described in Example I

T cell cultures 24 h after final stimulation with antigen armed DC were analyzed for interferon gamma production. Intracellular IFN-γ production by CD4+ and CD8+ T cells was measured by flow cytometry. Brefeldin A (10 μg/ml)(Pharmingen, San Diego, Calif.) was added to the T cell cultures 2 h after restimulation with antigen armed DC. After an additional 18 h of culture, cells were incubated with staining buffer (PBS with 1% human Ab serum) for 15 min at 4° C., washed again, pelleted and fluorochrome labeled antibodies to CD3, CD4, or CD8 (Becton Dickinson, San Jose, Calif.) added. The cells were fixed and permeabilized by incubation with perm/fix solution (Pharmingen, San Diego, Calif.) for 20 min on ice, washed and antibodies to IFNγ or appropriate isotype controls (Pharmingen, San Diego, Calif.) added. After incubation for 30 min on ice, the cells were washed, resuspended in staining buffer containing 2% paraformaldehyde and analyzed by flow cytometry.

Results demonstrated that the ex vivo combination of dendritic cells, taxol-induced apoptotic tumor cells, and binding agent together were superior in producing IFN-γ than dendritic cell alone or in combination with binding agent alone, or apoptotic tumor cell alone. Tumor cells, rendered apoptotic by Tazol treatment and combined with a binding agent prior to loading to DC were most potent in inducing CD8+ IFN-γ+ T cells. All four tested chemotherapeutic drugs, were more effective in inducing CTL than tumor cells rendered apoptotic by irradiation. Apoptotic tumor cells coated with the binding agent MAb-B43.13 prior to loading onto DC were more potent activators of CTL than apoptotic tumor cells alone or the binding agent alone for all apoptosis agents tested. Results are illustrated in FIG. 6.

Apoptosis/CTL Experiments

### Example VI

Twenty human patients diagnosed with recurrent ovarian cancer entered a study of non-radiolabeled murine MAb-B43.13 in combination with standard chemotherapeutic agents. Patients received twenty minute infusions of 2 mg of MAb-B43.13 at weeks 1, 3, 5, and 9, and a further optional dose at week 12. After treatment with MAb-B43.13, patients received standard chemotherapy and an optional dose between weeks 12 and 26 within 4 days of chemotherapy. Disease progression was assessed using CT scans, physical exam, CA125 levels, and long-term follow-up for survival. T cell responses to autologous tumor (n=8) and to CA125 (n=18) were assessed using ELISPOT assay for IFN-γ.

T Cell Responses

Patients peripheral blood mononuclear cells (PBMC) were thawed using standard techniques. The PBMC were allowed to sit for 2 minutes in the DNAse thaw media before washing. PBMC were washed once by first adding 8 mL AIM V media (commercially available from Gibco/Invitrogen Corporation, Carlsbad, Calif.). PBMC were resuspend in 10 mL AIM V media. 3-8×10⁶/mL PBMC in 10 ml AIM-V were incubated for one hour at 37° C., 5% CO₂ in a T75 flask plate.

After the incubation, the flask was washed with warm AIM V media four times (10 mL each wash), by adding the warm media to the side of the flask, not directly onto the adhered cells and decanting after each wash as well as aspirating the final wash.

After the final wash, Iscove's Modified Dulbecco's Media (10 mL IMDM commercially available from Gibco/Invitrogen Corporation, Carlsbad, Calif.), Fetal Bovine Serum (10% FBS commercially available from Gibco/Invitrogen Corporation, Carlsbad, Calif.), GM-CSF (1,000 U/ml), and IL-4 (1,000 U/ml) (both commercially available from R&D Systems, Minneapolis, Minn.) were added to the flask and incubated for 3 days at 37° C., 5% CO₂.

On day 3 the dendritic cell culture was fed by adding IMDM (2 mL), FBS (10%), GM-CSF (12,000,U), and IL-4 (12,000,U) to the flask (final cytokine concentration in flask was 1,000 U/ml GM-CSF, and 1,000 U/ml IL-4). Antibody and antigen were then complexed on day 6 for one hour at 37° C., 5% CO₂ in AIM-V. While complex was incubating, dendritic cells were harvested by tapping the flask after incubation with 4° C. PBS for 15 minutes at 4° C. Dendritic cells were then washed in plain AIM-V media (2-4 mL) and counted. A total of 25,000 to 100,000 dendritic cells were added to a 12 well plate. Antigen, antibody, antigen/antibody complex or controls were then added to each well and incubated in a total volume of 1 mL for 4 hours at 37° C., 5% CO₂.

Supernatant was removed and AIM-V (1 mL), TNF-α (10 ng/mL), IL-1β (10 ng/mL), and IL-6 (10 ng/mL) (commercially available from R&D Systems) was added to the culture and incubated overnight at 37° C., 5% CO₂.

The following day the in vitro stimulation was initiated by thawing patient T cells obtained from various time points (i.e., 12 weeks sample prior to chemotherapy and 26 week sample post chemotherapy). Cells were counted and resuspended with RPMI-1640 (1-2×10⁶ mL commercially available from Life Technologies, Frederick, Md.), FBS (10%), L-glutamine and gentamycin (commercially available from R&D Systems, Minneapolis, Minn.), IL-2 (20 IU/mL) and IL-7 (10 ng/mL).

Media was aspirated from the cultured dendritic cells and washed with AIM-V media. Patient T cells were then added at a ratio of 10-20:1 and incubated for 10 days at 37° C., 5% CO₂.

On day 10 the culture was fed with RPMI-1640 (0.5 mL), FBS (10%), L-glutainine and gentamycin, and IL-2 (80 IU/mL) and incubated for three days at 37° C., 5% CO₂.

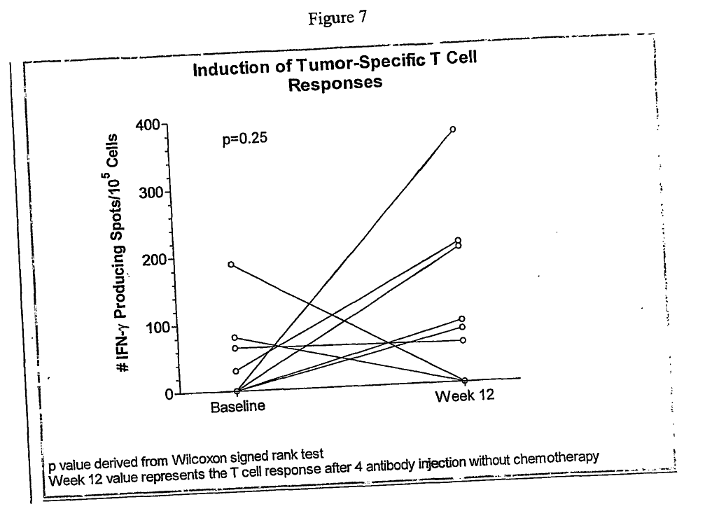

Results were analyzed using ELISPOT assay for T cell secretion of IFN-γ. Patients receiving non-radiolabeled MAb-B43.13 demonstrated increases in tumor-specific T cells post-administration of antibody alone (4 injections, week 12) as illustrated in FIG. 7. Similar T cell responses were seen for CA125. T cell samples taken at the 12 week time point (MAb-B43.13 administered prior to chemotherapy) had a lower T cell response to autologous tumor than patient samples taken at the 26 week time point (non-radiolabeled MAb-B43.13 administration in combination with chemotherapy) as illustrated in FIG. 7.

Beneficial Treatment Effect of T cell Responses

Using statistical analysis, time to progression and survival advantages were correlated with T cell responses to CA125 and/or autologous tumor. Patients that exhibited a T cell response to autologous tumor and/or CA125 and had a significant increase in time to progression (median not reached at the 108 week time point vs. 10.1 weeks, p<0.0001) as illustrated in the Kaplan Meier representation of FIG. 9A. Additionally, patients who exhibited a T cell response to autologous tumor and/or CA125 also had a significant increase in survival (median not reached at the 120 week time point vs. median of 51.9 weeks, p=0.0019) as illustrated in the Kaplan Meier representation of FIG. 9B.

Materials

MAb-B43.13 is a murine monoclonal IgG₁ antibody to CA125 (AltaRex Corp.). Chemotherapeutic agents (paclitaxel, doxorubicin, topotecan, carboplatin) were obtained from LKT Labs.

Cells

NIH:OVCAR-3 ovarian cancer cell line was purchased from ATCC (Manassas, Va.). Peripheral Blood Leukocytes (PBL) of healthy normal donors were obtained by leukaphoresis (SeraCare, Calif.) and purified on a Histopaque gradient (Sigma, Mississauga, Canada). Dendritic cells were prepared from normal human PBL by negative selection with anti-CD3, -CD7, -CD16, -CD19 and -CD56 followed by magnetic bead conjugated anti-mouse IgG and magnet separation (Monocyte isolation kit, Dynal), or by adherence. Cells were cultured in GM-CSF (1000 U/mL) and IL-4 (1000 U/mL) for 4 days to generate immature DC. DC were matured using TNF-α (50 U/mL) and IFN-α (10 ng/mL). T cells were purified from normal human PBL by negative selection using a T cell isolation kit (Dynal).

In vitro Activation of T Cells

NIH:OVCAR-3 tumor cells were induced to undergo apoptosis by irradiation (10,000 Rad), or with chemotherapeutic drugs (4-24 h incubation), washed, and fed to HLA-matched immature DC. In parallel, a set of apoptotic cells were incubated with MAb-B43.13 prior to loading of immature DC. As a control, necrotic NIH:OVCAR-3 cells (repeated freeze-thaw cycles) were fed to immature DC with and without MAb-B43.13. DC were loaded for 2 h at a ratio of tumor cells per DC, matured and incubated for 3 days. On day 7, DC were harvested and washed, and purified autologous T cells were added at a ratio of 10:1 ( cells to DC) and cultured for another 7 days. After one additional stimulation round with DC, the T cells were re-stimulated with loaded DC and controls for 24 h at a ratio of 20:1, followed by analysis for T cell activation.

Intracellular IFN-γ Staining

T Cells were incubated with brefeldin-A for 16-20 h after the final stimulation with loaded DC. Cells were stained with anti-CD3-FITC and anti-CD8-CyChrome, permeabilized and then stained with anti-IFN-γ-PE followed by flow cytometry analysis.

Chromium Release Assay

NIH:OVCAR-3 cells were labelled with ⁵¹Cr (˜100 μCi/2×10⁶ cells) for 2 h, then added to serial dilutions of activated T cells. After a 4-h incubation, plates were centrifuged and aliquots of supernatants analyzed for released ⁵¹Cr in a gamma counter. The percentage of specific lysis was calculated according to the formula: (Release in the presence of Activated T cells−Spontaneous Release)/(Maximum Release−Spontaneous Release)×100.

WST-1 for Monitoring Drug-Induced Cell Death

NIH:OVCAR-3 cells were grown in 96-well plates (NUNC) and irradiated with 10,000 rad or treated with chemotherapeutic drugs in a range of concentrations for 4 h, followed by washing. Cells were incubated at 3 7° C. for up to 3 days. WST-1 substrate (Boehringer-Mannheim, Mannheim, Germany) was added for 4 h 24, 48, and 72 h after treatment. Plates were read in an ELISA reader at 650 nm and the percentage of cell death calculated according to the formula: A650 of treated cells/A650 of untreated cells×100.

Annexin V Apoptosis Assay

NIH:OVCAR-3 cells were grown in 6-well plates (NUNC) and irradiated or treated with chemotherapeutic drugs for 4-48 h, washed and stained with Annexin V-FITC (BD-Pharmingen) for 1 h. Cells were analyzed by flow cytometry (Becton-Dickinson, CellQuest), counterstained with Propidium Iodide and analyzed again in the flow cytometer.

Confocal Microscopy for Activated Caspases

NIH:OVCAR-3 cells were grown in tissue chamber slides (NUNC) and treated with chemotherapeutic drugs for 4-48 h, washed and stained with Phi-Phi-Lux for 1 h. Cells were washed briefly again, fixed and counterstained with Propidium Iodide prior to analysis by confocal microscopy (Zeiss, Germany).

CA125 Expression

NIH:OVCAR-34 cells were analyzed for CA125 expression prior to and 24 h after apoptosis induction with chemotherapeutic drugs or irradiation. Cells were incubated on ice with FITC-labeled MAb-B43.13 (FITC labeling kit, Molecular Probes, Eugene Oreg.) at 5 μg/mL for 1 h, washed twice, fixed and analyzed by flow cytometry.

Dendritic Cell Uptake of Tumor Cell by Confocal Microscopy

Immature dendritic cells were grown in chamber slides and incubated for 4 h-72 h with CFSE-labeled tumor cells undergoing apoptosis with and without MAb-B43.13 opsonization. Cells were fixed, permeabilized and stained with DAPI and antibodies against toll-like receptors 2, 3 and 6, followed by anti-rabbit-PE.

1. A method for treating a patient to reduce proliferation of and/or kill target cells that express an antigen, comprising
(a) administering one or more agents that cause apoptosis of the target cells; and
(b) administering an antibody immunoreactive with said antigen, and
wherein said antibody is cytotoxic to said target cells.

2. The method of claim 1, wherein the target cells are transformed cells.

3. The method of claim 2, wherein the transformed cells are tumor cells.

4. The method of claim 1, wherein the treatment reduces the number of target cells in the patient.

5. The method of claim 1, wherein the agent that causes apoptosis and the antibody are administered to the patient conjointly.

6. The method of claim 1, wherein the antibody is administered to the patient after the agent that causes apoptosis.

7. The method of claim 1, wherein the antibody is administered to the patient prior to the agent that causes apoptosis.

8. The method of claim 1, wherein the one or more agents that cause apoptosis of the target cells is a chemotherapeutic agent.

9. The method of claim 1, wherein the antibody is a xenotypic monoclonal antibody.

10. The method of claim 9, wherein said xenotypic monoclonal antibody is selected from the group consisting of Alt-1, Alt-2, Alt-3, Alt-4, and Alt-5.

11. The method of claim 1, wherein the one or more agents that cause apoptosis and the antibody elicit an effective B and/or T cell response when administered to the patient.

12. The method of claim 11, wherein the effective T cell response is selected from the group consisting of a T helper response; a CTL response; and a T helper response and a CTL response.

13. The method of claim 1, wherein the patient is a human.

14. A packaged pharmaceutical for treating a patient to reduce proliferation of and/or kill target cells that express a antigen, comprising
(a) an antibody formulation immunoreactive with said antigen, which is accessible on target cells and said antibody formulation induces endocytosis of the target cell by an antigen presenting cell, and said antibody formulation is cytotoxic to said target cells; and
(c) instructions for using the antibody formulation in conjunction with a treatment that causes apoptosis of the target cells.

15. The packaged pharmaceutical of claim 14 further comprising one or more agents that cause apoptosis of the target cells.

16. The packaged pharmaceutical of claim 15, wherein the one or more agents that cause apoptosis of the target cells is a chemotherapeutic agent.

17. The packaged pharmaceutical of claim 15, wherein the one or more agents that cause apoptosis are formulated separately from the antibody formulation.

18. The packaged pharmaceutical of claim 15, wherein the one or more agents that cause apoptosis are formulated with the antibody formulation.

19. The packaged pharmaceutical of claim 14, wherein the antibody formulation is a xenotypic monoclonal antibody formulation.

20. The packaged pharmaceutical of claim 19, wherein said xenotypic monoclonal antibody formulation is selected from the group consisting of Alt-1, Alt-2, Alt-3, Alt-4, and Alt-5.

21. The packaged pharmaceutical of claim 14, wherein the target cell is a transformed cell.

22. The packaged pharmaceutical of claim 21, wherein the transformed cell is a tumor cell.

23. The packaged pharmaceutical of claim 14, wherein the one or more agents that cause apoptosis of target cells and the antibody formulation induce an effective B and/or T cell response in the patient.

24. The packaged pharmaceutical of claim 23, wherein the effective T cell response is selected from the group consisting of a T helper response; a CTL response; and a T helper response and a CTL response.

25. The pharmaceutical package of claim 14, wherein the antibody formulation is formulated at a dosage of about 100 μg/patient to about 2 mg/patient.

26. The pharmaceutical package of claim 14, wherein the antibody formulation is formulated at a dosage of about 0.1 μg/ml to about 200 μg/ml.

27. The pharmaceutical package of claim 14, wherein the antibody formulation is lyophilized.

28. A kit for treating a patient to reduce proliferation of and/or kill target cells that express a antigen, comprising
(a) one or more agents that cause apoptosis of the target cells ex vivo;
(b) an antibody formulation immunoreactive with said antigen, which is accessible on target cells and said antibody formulation induces endocytosis of the target cell by an antigen presenting cell, and said antibody formulation is cytotoxic to said target cells; and
(c) instructions for treating target cells ex vivo with said one or more apoptotic agent(s) and administering treated target cells conjointly with said antibody formulation.

29. The kit of claim 28, wherein said kit includes a means for isolating target cells from a patient sample.

30. The kit of claim 29, wherein the means for isolating target cells from a patient sample comprises an affinity purification means selected from the group consisting of an antibody; a lectin; a His-tag sequence; and an enterokinase cleavage tag.

31. The kit of claim 28, wherein said kit includes a means for isolating dendritic cells from a patient sample.

32. The kit of claim 28, wherein said kit includes HLA-matched dendritic cells.

33. The kit of claim 28, wherein the antibody is a xenotypic monoclonal antibody.

34. The kit of claim 33, wherein the xenotypic monoclonal antibody is selected from the group consisting of Alt-1; Alt-2; Alt-3; Alt-4; and Alt-5.

35. The kit of claim 28, wherein the one or more agents that cause apoptosis of the target cells ex vivo is a chemotherapeutic agent.
